(19) United States
(12) Patent Application Publication
(10) Pub. No.: US 2025/0302364 A1
(43) Pub. Date: Oct. 2, 2025

(54) SMART GARMENT

(71) Applicant: Myant Inc., Etobicoke (CA)

(72) Inventors: Milad Alizadeh-Meghrazi, Toronto (CA); Tony Chahine, Toronto (CA); Ladan Eskandarian, Toronto (CA); Christopher L. Swenglish, Connellsville, PA (US); Saundra L. Penkunas, Portland, OR (US); Brian M. Etzel, Butler, PA (US); Charles Amurgis, Mars, PA (US); Joyce Chow, Markham (CA); Tafarel Ribeiro, Toronto (CA); Trevor Hung, Markham (CA)

(21) Appl. No.: 19/235,480

(22) Filed: Jun. 11, 2025

Related U.S. Application Data
(63) Continuation of application No. PCT/US23/83923, filed on Dec. 13, 2023.
(60) Provisional application No. 63/432,465, filed on Dec. 14, 2022.

(51) Int. Cl. A61B 5/363; A61B 5/00; A61B 5/256; A61B 5/27; A61B 5/28; A61N 1/39

(52) U.S. Cl. CPC A61B 5/363; A61B 5/256; A61B 5/27; A61B 5/28; A61B 5/7405; A61N 1/3925

(57) ABSTRACT

A non-invasive, wearable, ambulatory device capable of cardiac defibrillation includes a plurality of ECG electrodes and a plurality of therapy electrodes. A smart garment is configured to be worn around a torso of the patient. The smart garment includes a first fabric portion and a multiaxially expandable fabric portion coupled with the first fabric portion. The multiaxially expandable fabric portion is configured to expand along a first axis as the multiaxially expandable fabric portion expands along a second axis. The plurality of ECG electrodes are coupled with the multiaxially expandable portion, the first fabric portion, or both the multiaxially expandable fabric portion and the first fabric portion. The smart garment is configured to maintain continuous electrical contact between the plurality of ECG electrodes and skin of the patient while the smart garment is worn about the torso of the patient.

### CROSS REFERENCE TO RELATED APPLICATIONS

This patent application is a continuation of PCT patent application no. PCT/US2023/083923, filed Dec. 13, 2023, and entitled “SMART GARMENT,” which in turn claims priority from U.S. provisional patent application No. 63/432,465, filed on Dec. 14, 2022, the disclosures of which are each incorporated herein, in their entirety, by reference.

### FIELD IN THIS DISCLOSURE

Illustrative embodiments in this disclosure generally relate to smart garments, including smart garments for physiological monitoring.

### BACKGROUND

Sensory devices, such as physiological data sensors, may be integrated or embedded into garments. As an example, smart garments may be used for medical applications, such as for wearable cardioverter defibrillators. Smart garments may also be used to help with monitoring and improving athletic performance. When sensory devices are embedded into garments, the sensory devices may be positioned physically proximate to user limbs or body parts. The garments having the sensory devices embedded therein may be worn by users for extended durations of time.

### SUMMARY

In accordance with an embodiment, a non-invasive, wearable, ambulatory device capable of cardiac defibrillation includes a plurality of ECG electrodes and associated ECG circuitry configured to sense ECG signals from a patient. A plurality of therapy electrodes are configured to deliver one or more defibrillation pulses to the patient. A smart garment is configured to be worn around a torso of the patient. The smart garment includes a first fabric portion and a multiaxially expandable fabric portion coupled with the first fabric portion. The multiaxially expandable fabric portion is configured to expand along a first axis as the multiaxially expandable fabric portion expands along a second axis. The first axis and the second axis are substantially normal to one another. The plurality of ECG electrodes are coupled with the multiaxially expandable portion, the first fabric portion, or both the multiaxially expandable fabric portion and the first fabric portion. The smart garment is configured to maintain continuous electrical contact between the plurality of ECG electrodes and skin of the patient over a duration of time when the smart garment is worn about the torso of the patient. In one or more examples, a smart garment may comprise a garment having: one or more electrodes attached to or otherwise incorporated therein for contacting the wearer; one or more sensors attached to or incorporated therein for obtaining data from the wearer; one or more processors attached to or incorporated therein for processing information about the wearer of the garment; and/or one or more power sources attached to or incorporated therein for powering the one or more sensors, if present, and one or more processors, if present. In one or more examples, a smart garment may comprise a garment incorporating one or more textiles that facilitates the integration of electronic components (e.g., electrodes, sensors, and/or processors) into the garment.

Among other things, the plurality of ECG electrodes may include a plurality of non-adhesive ECG electrodes. The plurality of ECG electrodes may include a plurality of dry ECG electrodes. The plurality of ECG electrodes may be polarizable ECG electrodes.

In various embodiments, the smart garment is configured to maintain continuous electrical contact between the plurality of therapy electrodes and skin of the patient over the duration of time when the smart garment is worn about the torso of the patient. A controller may be configured to detect ventricular fibrillation (“VF”) and to deliver one or more defibrillation pulses to the patient via the plurality of therapy electrodes. Additionally, or alternatively, the controller may be configured to detect ventricular tachycardia (“VT”) and to deliver one or more defibrillation pulses to the patient via the plurality of therapy electrodes. In various embodiments, the one or more defibrillation pulses may include transcutaneous defibrillation pulses. The defibrillation pulses may be between 25 joules and 400 joules. In some embodiments, the pulses are cardioversion pulses. The energy of one or more of the cardioversion pulses may be between 25 joules and 400 joules.

Among other things, the controller may be configured to detect one or more of bradycardia, tachycardia, or asystole, and to deliver one or more pacing pulses to the patient via the plurality of therapy electrodes. The one or more pacing pulses may include transcutaneous pacing pulses. the current of one or more of the pacing pulses may be between 0.1 mA and 300 mA.

In some embodiments, the first fabric portion is configured to cause the smart garment to maintain electrical contact between one or more of the plurality of ECG electrodes and skin of a patient when the smart garment is subject to forces that cause the smart garment to either one of (a) stretch along, or (b) twist about, a circumference of the patient's body in an anatomical axial plane. In a similar manner, the smart garment may also be configured to maintain electrical contact between the plurality of therapy electrodes and skin of a patient when the smart garment is subject to forces that cause the smart garment to either one of (a) stretch along, or (b) twist about, a circumference of the patient's body.

The smart garment may also be configured to maintain the electrical contact between one or more of the plurality of ECG electrodes and skin of the patient at least by pressing the one or more of the plurality of ECG electrodes against the skin of the patient of the smart garment within a pressure of between about 0.1 psi and about 3 psi. In a similar manner, the smart garment may be configured to maintain the electrical contact between the one or more of the plurality of therapy electrodes and skin of the patient at least by pressing the one or more of the plurality of therapy electrodes against the skin of the patient of the smart garment at a pressure of between about 0.1 psi and about 3 psi.

In some embodiments, the controller is configured to trigger an audible alarm when an arrhythmia is detected. Additionally, or alternative, the controller may be configured to trigger a tactile alarm when an arrhythmia is detected.

The multiaxially expandable fabric portion may have a negative Poisson's ratio, and the first fabric portion may have a positive Poisson's ratio. The multiaxially expandable fabric portion may be formed of a matrix a plurality of single cells. Each of the single cells may have a predetermined shape.

In various embodiments, each of the plurality of ECG electrodes includes a smart garment coupling portion. The smart garment may include a plurality of corresponding ECG electrode coupling portions. The coupling portions are configured to couple the plurality of ECG electrodes with the smart garment. Each of the smart garment coupling portions may have a color identification. Each of the ECG electrode coupling portions may have a corresponding color identification.

In some embodiments, the plurality of ECG electrodes are removably coupled with the smart garment. Alternatively, the plurality of ECG electrodes may be permanently coupled with the smart garment.

The smart garment may include two shoulder straps configured to be worn over the shoulders of the patient. Additionally, the smart garment may include a belt configured to be worn about a torso region of the patient's body. In various embodiments, the plurality of ECG electrodes are coupled with the belt. The belt may have the first fabric portion and the multiaxially expandable fabric portion. The multiaxially expandable fabric portion may be adjacent to one or more of the plurality of ECG electrodes. Additionally, or alternatively, the multiaxially expandable fabric portion may be configured to be proximate to one or more chest ECG electrodes of the plurality of ECG electrodes. The first fabric portion may surround the multiaxially expandable fabric portion, and/or overlap the multiaxially expandable fabric portion. The multiaxially expandable fabric portion may be configured to be proximate to at least one therapy electrode.

Among other things, the smart garment may include one or more pockets configured to receive the plurality of therapy electrode. The smart garment may have a back portion having one or more of the pockets. The pockets may include at least a portion formed from the multiaxially expandable fabric portion. The multiaxially expandable fabric portion may include a multiaxially expandable fiber, multiaxially expandable fabric yarn, or both a multiaxially expandable fiber and a multiaxially expandable fabric yarn. The multiaxially expandable fabric yarn includes a double helix yarn. A wrap material of the double helix yarn may include an ultra-high molecular weight polyethylene fiber and a core material is polyurethane. Such ultra-high molecular weight polyethylene fiber may have a molecular mass between about 3.5 million and about 7.5 million amu.

The multiaxially expandable fabric portion may include a fiber-based non-conductive yarn having a multiaxially expandable fabric structural unit. The fiber-based yarn has a reentrant hexagonal structural unit in an unstretched configuration, and a honeycomb structural unit in a stretched configuration, wherein the fiber-based yarn is lengthened between 5% and 25% in the stretched position relative to the unstretched position. The multiaxially expandable fabric portion may be formed from a single layer or double layer multiaxially expandable fabric material.

The multiaxially expandable fabric portion may be formed from an additive printing process. The multiaxially expandable fabric portion may include a woven fabric portion. The multiaxially expandable fabric portion may include a knitted fabric portion formed using a knitting machine. The knitting machine may be a circular knitting machine, flat-bed knitting machine, or a V-bed knitting machine.

In various embodiments, the multiaxially expandable fabric portion may also be a multiaxially contractible portion. Among other things, the expandable fabric portion may be a biaxially expandable fabric portion or a triaxially expandable fabric portion. The first axis, the second axis may be normal to one another. Additionally, a third axis may be normal to the first axis and the second axis. The multiaxially expandable fabric portion may expand and/or contract simultaneously along two or more of the aforementioned axes.

In accordance with another embodiment, a smart garment for using in monitoring includes a first fabric portion and a multiaxially expandable fabric portion. The multiaxially expandable portion has a first axis and a second axis normal to one another. The multiaxially expandable fabric portion is configured to expand along the first axis as the multiaxially expandable fabric portion expands along the second axis. The first fabric portion is coupled with the multiaxially expandable fabric portion. One or more electrodes are coupled with the multiaxially expandable fabric portion, the first fabric portion, or both the multiaxially expandable fabric portion and the first fabric portion.

In various embodiments, the smart garment may be used for cardiac health monitoring. Accordingly, the electrodes may be ECG electrodes

In various embodiments, the smart garment is thereby configured to maintain continuous electrical contact between the plurality of ECG electrodes and skin of the patient over a duration of time when the smart garment is worn about the torso of the patient. Thus, in one or more examples, the use of multiaxially expandable fabric portion may at least in part effectively maintain continuous electrical contact between the plurality of ECG electrodes and skin of the patient

Illustrative embodiments in this disclosure are implemented as a computer program product having a computer usable medium with computer readable program code thereon. The computer readable code may be read and utilized by a computer system in accordance with conventional processes.

### BRIEF DESCRIPTION OF THE DRAWINGS

Various aspects of at least one example are discussed below with reference to the accompanying figures, which are not intended to be drawn to scale. The figures are included to provide an illustration and a further understanding of the various aspects and examples, and are incorporated in and constitute a part of this specification, but are not intended to limit the scope of the disclosure. The drawings, together with the remainder of the specification, serve to explain principles and operations of the described and claimed aspects and examples. In the figures, each identical or nearly identical component that is illustrated in various figures is represented by a like numeral. For purposes of clarity, not every component may be labeled in every figure.

FIG. 1 schematically shows a user wearing a smart garment in accordance with illustrative embodiments in this disclosure.

FIG. 2A schematically shows medical devices that may be coupled to and/or integrated with the smart garment in accordance with illustrative embodiments in this disclosure.

FIG. 2B schematically shows an alternative medical device that may be coupled to and/or integrated with the smart garment in accordance with illustrative embodiments in this disclosure.

FIG. 3 schematically shows a patient wearing the smart garment having the medical devices in accordance with illustrative embodiments.

FIG. 4A schematically shows an example smart garment in accordance with illustrative embodiments.

FIG. 4B schematically shows a magnified view of a portion of the smart garment of FIG. 4A.

FIG. 4C schematically shows a magnified view of another portion of the smart garment of FIG. 4A.

FIG. 4D schematically shows the back of the portion of the smart garment of FIG. 4C.

FIG. 5A schematically shows a biaxially expandable fabric portion in accordance with illustrative embodiments in this disclosure.

FIG. 5B schematically shows the biaxially expandable fabric portion of FIG. 5A in a compressed configuration.

FIG. 6A schematically shows details of the biaxially expandable fabric portion in accordance with illustrative embodiments in this disclosure.

FIG. 6B schematically shows the single cell when the biaxially expandable fabric portion is in a resting configuration in accordance with illustrative embodiments in this disclosure.

FIG. 6C schematically shows the single cell when the biaxially expandable fabric portion is in the stretched configuration controller in accordance with illustrative embodiments in this disclosure.

FIG. 6D shows the biaxially expandable fabric portion in the resting configuration controller in accordance with illustrative embodiments in this disclosure.

FIG. 6E shows the biaxially expandable fabric portion in the stretched configuration controller in accordance with illustrative embodiments in this disclosure.

FIG. 7A schematically shows a triaxially expandable fabric portion in accordance with illustrative embodiments in this disclosure.

FIG. 7B schematically shows the triaxially expandable fabric portion of FIG. 7A in a compressed configuration.

FIG. 8A schematically shows details of the triaxially expandable fabric portion in accordance with illustrative embodiments in this disclosure.

FIG. 8B schematically shows a top view of the single cell when the triaxially expandable fabric portion is in the resting configuration in accordance with illustrative embodiments in this disclosure.

FIG. 8C schematically shows the top view of the single cell when the triaxially expandable fabric portion is in the stretched configuration in accordance with illustrative embodiments in this disclosure.

FIG. 8D schematically shows a side view of the cell of FIG. 8B.

FIG. 8E schematically shows a side view of the cell of FIG. 8C.

FIG. 9 schematically shows a multi-layer expandable fabric portion in accordance with illustrative embodiments in this disclosure

FIG. 10A schematically shows a knitting pattern for a multiaxially expandable fabric portion in accordance with illustrative embodiments.

FIG. 10B shows a picture of the knitted fabric portion in the resting configuration using the knitting pattern of FIG. 10A.

FIG. 10C shows a picture of the knitted fabric portion of FIG. 10B in the stretched configuration.

FIG. 11A schematically shows a knitting pattern for an alternative multiaxially expandable fabric portion in accordance with illustrate embodiments.

FIG. 11B shows a picture of the knitted fabric portion in the resting configuration 118R using the knitting pattern of FIG. 11A.

FIG. 11C shows a picture of the knitted fabric portion of FIG. 11B in the stretched configuration.

FIG. 12 schematically shows a controller of the smart garment in accordance with illustrative embodiments in this disclosure.

### DETAILED DESCRIPTION

This disclosure relates to techniques, processes, and devices implementing multiaxially expandable fabric portions in smart garments, including for medical applications. In illustrative embodiments, a smart garment as disclosed herein includes a multiaxially expandable (e.g., biaxially expandable or triaxially expandable) fabric portion configured to expand along at least one axis (or orientation) when subject to force or pressure causing it to expand along a different axis (or orientation). For example, the axes may be substantially normal (e.g., orthogonal or perpendicular) to one another. For example, a biaxially expandable fabric portion is configured to expand along a first axis when subject to force or pressure causing it to expand along a second axis. In implementations, the first and second axis are perpendicular to one another. For example, a triaxially expandable fabric portion is configured to expand along first and second axes when subject to force or pressure causing it to expand along a third axis. In implementations, the first, second, and third axis may be substantially normal (e.g., orthogonal or perpendicular) to one another. In implementations herein, such multiaxially expandable fabric portions can be disposed within, e.g., integrated into smart garment fabric. For example, such multiaxially expandable fabric portions can be deployed in predetermined regions of the smart garment as explained in further detail below. In various embodiments, the multiaxially expandable fabric portion is also a multiaxially contractible portion (e.g., along the same axes along which the portion is multiaxially expandable). These forces or pressures applied to the expandable fabric portion can be introduced during movement of the garment wearer, e.g. the body of the wearer and the garment can experience relative movement due to daily physical activities of the wearer (walking, sleeping, etc.).

When wearing garments with embedded or otherwise attached sensors, one problem that can be encountered is relative movement between the sensor and the body of the garment wearer. In these situations, it is understood that calibration of the sensor can be affected. Further, sensitivity of the sensor can be negatively affected when the sensor is permanently or intermittently dislodged from a selected position on the body of the wearer. It is common for sensors to become displaced away from the selected position during physical activity or movement of the wearer.

In one implementation, the multiaxially expandable fabric portion is positioned adjacent to a sensor, actuator, and/or therapy device (collectively the “device”) that may be disposed, e.g., integrated, in the smart garment. Such devices can include, for example, ECG electrodes, therapy electrodes, vibration actuators, cardiovibration sensors, or sensor panels comprising one or more of a combination for the foregoing devices. In this regard, the multiaxially expandable fabric portion helps maintain an appropriate position, and/or orientation, and/or pressure range of contact of the device relative to the patient's skin during the ordinary, everyday, routine, and/or prescribed course of use of the smart garment. Illustrative embodiments thus provide a more reliable coupling between the user and the device of the smart garment. This may be particularly advantageous for medical applications, such as for wearable cardioverter defibrillators that have integrated ECG sensors and therapy electrodes. Details of illustrative embodiments are discussed below.

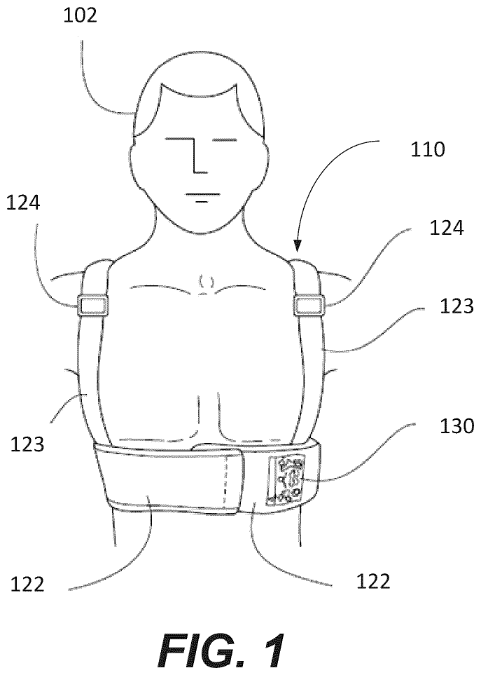

FIG. 1 schematically shows a user 102 (e.g., patient 102) wearing a smart garment 110 in accordance with illustrative embodiments in this disclosure. Among other uses, the smart garment 110 may include a wide variety of electronic and mechanical devices for monitoring and treating patients' 102 medical conditions. In some examples, depending on the underlying medical condition being monitored or treated, devices such as cardiac defibrillators may be externally connected to the patient 102. In some cases, physicians may use devices alone or in combination with drug therapies to treat conditions such as cardiac arrhythmias.

The smart garment 110 may be provided in the form of a vest or harness having a back portion and sides extending around the front of the patient 102 to form a belt 122. The ends of the belt 122 are connected at the front of the patient 102 by a closure, which may comprise one or more clasps. Multiple corresponding closures may be provided along the length of the belt 122 to allow for adjustment in the size of the secured belt 122 in order to provide a more customized fit to the patient 102. The smart garment 110 may further include two straps 123 connecting the back portion to the belt 122 at the front of the patient 102. The straps 123 have an adjustable size to provide a more customized fit to the patient 102. The straps 123 may be provided with sliders 124 to allow for the size adjustment of the straps 123. The straps 123 may be removably attached to the belt 122 at the front of the patient 102. In implementations, the straps 123 may be permanently secured to the belt 122 such that straps 123 cannot be separated from the belt without destroying the garment 110.

The smart garment 110 may include an elastic, low spring rate material that stretches appropriately to keep the device (e.g., electrodes) in place against the patient's 102 skin while the patient 102 moves. To that end, the smart garment 110 may include a multiaxially expandable fabric portion. Preferably, the material of the smart garment 110 is lightweight and breathable. For example, the smart garment 110 may have elastic, low spring rate material composition based on a fiber content of about 10-30% (e.g., 20%) elastic fiber, 15-40% (e.g., 32%) polyester fiber, and about 0-60% (e.g., up to 48%) or more of nylon or other fiber. Additionally, the smart garment 110 may include multiaxially expandable fabric in one or more portions of the smart garment 110 in accordance with examples described herein.

In accordance with one or more examples, the smart garment 110 and/or multiaxially expandable fabric portions thereof may be formed from an elastic, low spring rate material and constructed using tolerances that are considerably closer than those customarily used in garments. The materials for construction are chosen for functionality, comfort, and biocompatibility. The materials may be configured to wick perspiration from the skin. The smart garment 110 may be formed from one or more blends of nylon, polyester, and spandex fabric material. Different portions or components of the smart garment 110 may be formed from different material blends depending on the desired flexibility and stretchability of the smart garment 110 and/or its specific portions or components. For instance, the belt 122 of the smart garment 110 may be formed to be more stretchable than the back portion. According to one example, the smart garment 110 is formed from a blend of nylon and spandex materials, such as a blend of between 50-85% (e.g., 77%) nylon and 15-50% (e.g., 23%) spandex. According to another example, the smart garment 110 is formed from a blend of nylon, polyester, and spandex materials, such as 40% nylon, 32% polyester, and 14% spandex. According to another example, the smart garment 110 is formed from a blend of polyester and spandex materials, such as 86% polyester and 14% spandex or 80% polyester and 20% spandex. For example, the nylon and spandex material is configured to be aesthetically appealing, and comfortable, e.g., when in contact with the patient's skin. Stitching within the smart garment 110 may be made with industrial stitching thread. According to one example, the stitching within the smart garment 110 is formed from a cotton-wrapped polyester core thread. In various embodiments, the above mentioned materials may be formed as, or coupled to, multiaxially expandable fabric portions that assist with maintaining contact of the device with the user 102. Maintaining proper contact between the device (e.g., ECG electrodes, therapy electrodes, and/or the connection pod) and the user 102 is particularly important in medical applications, as discussed below.

In various embodiments, the smart garment 110 may include a dock 130 configured to receive an electronic device, such as the connection pod as described in further detail herein. In some embodiments, the dock 130 is attached to the garment 110 and includes circuitry and connectors configured to couple certain garment-based devices, such as, ECG electrodes that may be permanently integrated in the garment, to the connection pod when the connection pod is attached to the dock 130. For example, integrated wiring disposed within the fabric of the garment can be coupled from the ECG electrodes to one or more connector in the dock 130. These connectors can then facilitate the electrical communication of raw ECG signals from the plurality of ECG electrodes to the ECG acquisition and processing circuitry disposed within the connection pod.

One of the most deadly cardiac arrhythmias is ventricular fibrillation, which occurs when normal, regular electrical impulses are replaced by irregular and rapid impulses, causing the heart muscle to stop normal contractions and to begin to quiver. Normal blood flow ceases, and organ damage or death can result in minutes if normal heart contractions are not restored. Because the victim has no perceptible warning of the impending fibrillation, death often occurs before the necessary medical assistance can arrive. Other cardiac arrhythmias can include excessively slow heart rates known as bradycardia or excessively fast heart rates known as tachycardia. Cardiac arrest can occur when a patient in which various arrhythmias of the heart, such as ventricular fibrillation, ventricular tachycardia, pulseless electrical activity (PEA), and asystole (heart stops all electrical activity) result in the heart providing insufficient levels of blood flow to the brain and other vital organs for the support of life.

Cardiac arrest and other cardiac health ailments are a major cause of death worldwide. Various resuscitation efforts aim to maintain the body's circulatory and respiratory systems during cardiac arrest in an attempt to save the life of the patient. The sooner these resuscitation efforts begin, the better the patient's chances of survival. Ventricular fibrillation or ventricular tachycardia can be treated by an external defibrillator, for example, by providing a therapeutic shock to the heart in an attempt to restore normal rhythm. To treat conditions such as bradycardia, an external pacing device can provide pacing stimuli to the patient's heart until intrinsic cardiac electrical activity returns. The smart garment 110 includes features that can monitor for and treat such conditions.

This disclosure relates to smart garments 110 that incorporate devices, such as those described above. In particular, the disclosure relates to a smart garment 110 including a multiaxially expandable fabric that simultaneously expands at least along two substantially normal (e.g., substantially orthogonal or substantially perpendicular) axes, e.g., when stretched. Similarly, the multiaxially expandable fabric may simultaneously contract along at least the same two substantially normal axes, e.g., when released from a stretched position.

Advantageously, the multiaxially expandable fabric provides enhanced comfort to a user wearing the smart garment during wearing and movement, as the multiaxially expandable fabric expands and/or contracts in response to various user sizes and user movements. In contrast to consumer clothing that is manufactured with corresponding sizes (e.g., Large, Medium, Small, etc.), smart garments 110 incorporating medical devices frequently come in one size. Accordingly, various embodiments advantageously provide a high-performance smart garment for users of various sizes. Furthermore, the multiaxially expandable fabric maintains the device, such as an ECG and/or therapy electrodes, in a desired contact with the user while remaining in a desired location and orientation.

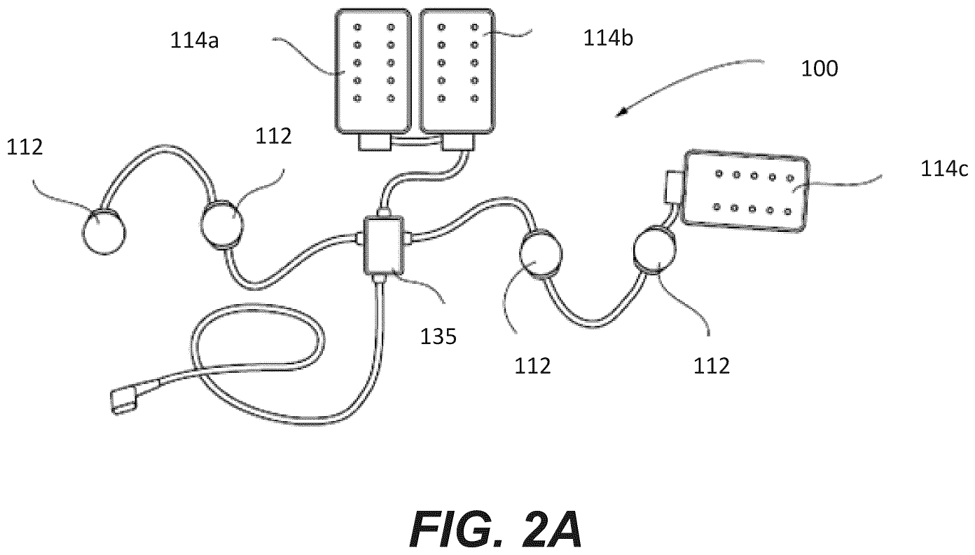

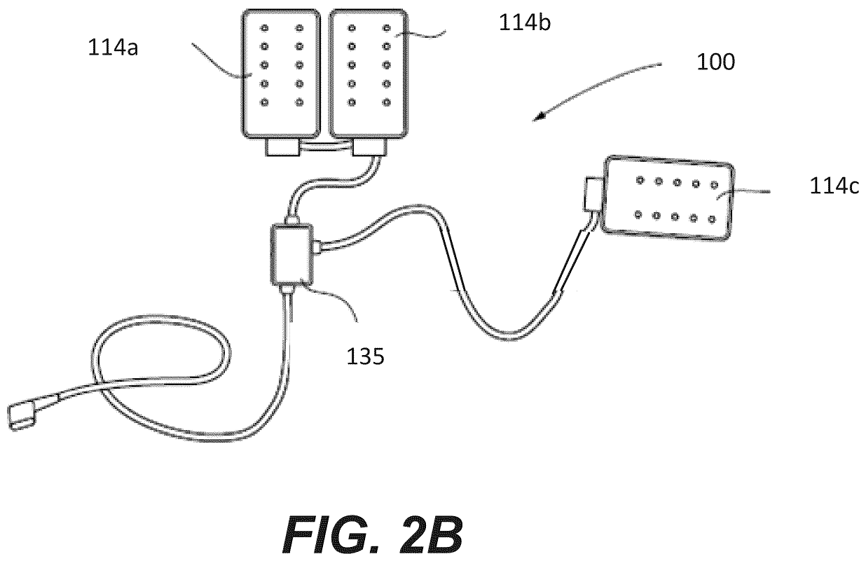

FIG. 2A schematically shows the medical device 100 (e.g., ECG electrodes 112 and/or therapy electrodes 114) that may be coupled to and/or integrated with the smart garment 110. As mentioned above, the multiaxially expandable fabric portion maintains the device 100, such as ECG electrodes 112 and/or therapy electrodes 114, in a desired contact with the user 102. For example, for ECG electrodes 112 to accurately detect ECG signals from the user 102, the ECG electrodes 112 should be in contact with the user's 102 skin. Problems frequently encountered with smart garments 110 having sensing electrodes 112 include electrode flipping (i.e., the electrode 112 contact surface becomes at least partially inverted, losing contact with the user's 102 skin) and mispositioning, as example embodiments of relative displacement experienced between the sensing electrodes 112 and the user's 102 skin/body. Various embodiments provide multiaxially expandable fabric adjacent to the electrodes 112 to reduce the likelihood of electrode 112 flipping or mispositioning. In a similar manner, the smart garment may be configured to include multiaxially expandable fabric adjacent to the therapy electrodes 114 to assist with maintaining a desired positioning and contact a with the user's 102 skin. Various embodiments help reduce ECG electrode 112 and/or therapy electrode 114 falloff.

To obtain a reliable ECG signal so that the monitor can function effectively and reliably, it is desirable for the sensing electrodes 112 to be in the proper position and in good contact with the patient's 102 skin. Preferably, the electrodes 112 remain in a substantially fixed position and preferably do not move excessively or lift off the skin's surface. As such, the ECG signal is not adversely affected with noise and is able to perform arrhythmia detection in the ECG analysis and monitoring system. Additionally, false alarms and/or shocks may be inhibited.

Similarly, to effectively deliver the defibrillating energy, it is desirable that the therapy electrodes, e.g., two rear therapy electrodes 114a and 114b, and a front therapy electrode 114c (collectively therapy electrodes 114) are in a proper position, orientation, and in appropriate range of contact pressure with the patient's skin. It is desirable for the therapy electrodes 114 to be firmly positioned against the skin, minimizing electrode-skin impedance, leading to an effective and/or efficacious delivery of transcutaneous therapeutic energy to the patient's heart. Also, properly positioned therapy electrodes 114 can minimize or eliminate damage to the patient's 102 skin, such as burning, when the shock is delivered.

FIG. 2B schematically shows an implementations of the medical device 100 that may be coupled to and/or integrated with the smart garment 110 in accordance with illustrative embodiments in this disclosure. In some embodiments, the smart garment 110 may include integrated ECG electrodes 112 that are not removable from the garment 110. Accordingly, electrical cables, wires, and/or fibers may be disposed within, embedded within, sewn onto, and/or printed onto, the garment 110 and may extend from various ECG electrodes 112 to the dock 130. The connection pod 135 may be configured to securably and releasable couple with the dock 130, such that the connection pod 135 is electrically coupled and communicates with the ECG electrodes 112 integrated in the garment 112. The connection pod 135 may be received directly into the receptacle of the dock 130. It is also recognized that the electrodes 112 can be incorporated into a body of the garment 110 as knit and/or woven structures (e.g. the sensors 112 are composed of conductive threads, which are electrically connected to the dock 130—see FIG. 1 and/or connection pod 135—see FIG. 2A).

In various embodiments, the connection pod 135 communicates with the controller 120, and establishes communication between the controller 120 and the various medical devices (e.g., electrode array 100). To that end, the connection pod 135 may include an analog-to-digital converter that receives analog signals from the ECG electrodes 112 and converts them to digital signals. The ECG signals (e.g., converted to digital) are forwarded to the controller 120 for further processing. Additionally, the controller 120 may forward a signal to the connection pod 135 to activate the release of an impedance-reducing gel from the therapy electrodes 114 and/or to initiate therapy delivery via the therapy electrodes 114. Additionally or alternatively, the controller 120 may also send signals to the connection pod 135 that notify the patient 102 via tactile stimulation or sensation (e.g., vibration) on skin of the patient, before a shock is delivered by the therapy electrodes 114. To that end, the connection pod 135 may also include an electromechanical motor therein under control of the controller 120 to effectuate the vibration. As noted herein, the connection pod 135 may be a device configured to be pressed up against skin of the patient to maximize likelihood of patient discerning the tactile stimulation or sensation on patient's skin.

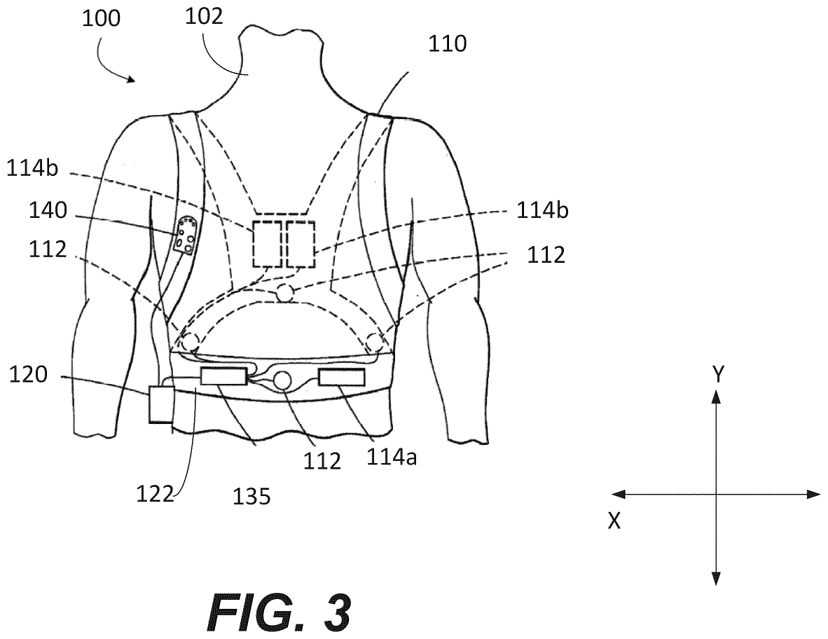

FIG. 3 schematically shows the patient 102 wearing the smart garment 110 in accordance with illustrative embodiments. The smart garment 110 may include one or more of the medical devices 100 described with reference to FIG. 2A, FIG. 2B, or a similar system. As such, the smart garment 110 may be configured as non-invasive, wearable, ambulatory device capable of cardiac defibrillation. The smart garment 110 may be capable of and designed for moving with the patient 102 as the patient 102 goes about his or her daily routine. In one example scenario, the wearable smart garment 110 can be worn nearly continuously or substantially continuously for an extended period of time, e.g., long term use comprising, longer than 2 weeks, about a month, or about two to three months, or about three to six months, at a time. During the period of time in which the garment 110 is worn by the patient 102, the wearable defibrillator can be configured to continuously or substantially continuously monitor the vital signs of the patient 102 and, upon determination that treatment is required, can be configured to deliver one or more therapeutic electrical pulses to the patient 102. For example, such therapeutic shocks can be pacing, defibrillation, cardioversion, or transcutaneous electrical nerve stimulation (TENS) pulses.

The smart garment 110 may include various devices 100, as described earlier, including, the one or more sensing electrodes 112 (e.g., ECG electrodes), one or more of the therapy electrodes 114a and 114b (collectively referred to herein as therapy electrodes 114), a controller 120, a connection pod 135, a patient interface pod 140 (e.g., having a button), a belt 122, or any combination of these. In some examples, at least some of the devices and/or physical components of the smart garment 110 can be configured to be affixed or attached to the garment 110 (or in some examples, permanently integrated into the garment 110—e.g. knit, woven or otherwise to or otherwise within the body of the garment), which can be worn about the patient's 102 torso.

In various embodiments, the controller 120 is configured to detect a treatable arrhythmia in the patient, and in response to such detection, initiate a treatment sequence or treatment protocol. For example, such a treatment sequence or treatment protocol begins with subtle notifications to the patient 102 and steadily escalates if the patient does not respond to such notifications in a timely manner, e.g., by providing additional audible and/or tactile and/or visual notifications to the patient 102. The smart garment 110 is configured to use a combination of low volume and high volume sirens, verbal messages, and/or flashing visual notifications to get the patient's 102 attention. As the wearable defibrillator device 100 of the smart garment 110 is designed to allow patients to return to most their normal daily activities with the peace of mind that they have protection from SCA death, the smart garment 110 is configured to provide easy access to under interface functionality to allow patients 102 to respond to alerts. The smart garment 110 does not require the assistance of another person or emergency personnel for it to work. The smart garment 110 can protect patients 102 even when they are alone. In a typical situation, the entire event, from detecting a life-threatening rapid heartbeat to automatically delivering a shock, may occur in about less than one minute.

As noted, in the course of the event, a feature of various embodiments of the smart garment 110 is the series of alerts and voice prompts that keep patients 102 informed about what the device 100 is doing. These alerts let patients 102 know that the device 100 is working to protect the patient. For example, in treating a life threatening event called a ventricular fibrillation (VF) where the patient does not respond to the alarms, the treatment process may proceed in the following manner. Initially, the arrhythmia is detected, activating a vibration alert to get the patient's attention. After around 5 seconds, if the patient doesn't respond, the controller 120 initiates an audible siren alarm. For the next 20 seconds, the controller 120 sirens get louder, and the controller 120 provides audible prompts instructing the patient to “Press response buttons”. At around 30-45 seconds from the onset of the arrhythmia, if the patient still hasn't responded, the wearable defibrillator device 100 proceeds to provide a treatment shock.

In connection with the above notification sequence, in response to detecting the treatable arrhythmia, the controller 120 can send a signal to a microcontroller disposed in the connection pod 135. In response, the microcontroller in the connection pod 135 can cause a vibration motor to begin vibrating to indicate to the patient 102 that a shock is imminent. To suspend or terminate an accidental or undesirable shock, the patient 102 may engage the patient interface pod 140 or press response buttons disposed on the controller 120. In some embodiments, the patient interface pod 140 may be coupled to the smart garment 110. In some other embodiments, the patient interface pod 140 may be integrated into the controller 120, or elsewhere.

The controller 120 can be operatively coupled to the sensing electrodes 112, which can be affixed to the garment 110, e.g., assembled into the garment 110 or removably attached to the garment 110, e.g., using hook and loop fasteners. In some implementations, the sensing electrodes 112 can be permanently integrated into the garment 110 (e.g., non-removable without destruction of the garment 110). However, in some other embodiments, the sensing electrodes 112 may be positioned with the garment 110 (e.g., by the user 102). The controller 120 can be operatively coupled to the therapy electrodes 114. For example, the therapy electrodes 114 can also be assembled into the garment 110, or, in some implementations, the therapy electrodes 114 can be permanently integrated into the garment 110. Sensing electrodes 112 and therapy electrodes 114 can also generically be referred to as sensors 112.

Component configurations other than those shown in FIG. 1 are possible. For example, the sensing electrodes 112 can be configured to be attached at various positions about the body of the patient 102. The sensing electrodes 112 can be operatively coupled to the controller 120 through the connection pod 135. In some implementations, the sensing electrodes 112 can be adhesively attached to the patient 102. In some implementations, the sensing electrodes 112 and at least one of the therapy electrodes 114 can be included on a single integrated patch and adhesively applied to the patient's 110 body.

The sensing electrodes 112 can be configured to detect one or more cardiac signals. Examples of such signals include ECG signals and/or other sensed cardiac physiological signals from the patient 102. In certain implementations, the sensing electrodes 112 can include additional components such as accelerometers, acoustic signal detecting devices, and other measuring devices for recording additional parameters. For example, the electrode 112 surfaces can be based on stainless steel, noble metals such as platinum, or Ag—AgCl. In an example scenario, a dry metal substrate can be placed directly on the skin and, as a result of the contact between the substrate and the skin, perspiration can accumulate on the substrate surface to provide electrical coupling with skin of the patient. In this regard, a dry substrate can be constructed from a housing configured to hold various circuit components and a treated, anodized metal surface configured to contact the patient's skin. For example, the treated, anodized metal surface can be treated with a tantalum pentoxide coating. In some examples, the sensing electrodes 112 can be used with an electrolytic gel dispersed between the electrode surface and the patient's skin. In implementations, advantages of dry ECG electrodes as sensing electrodes 112 include a benefit of not needing an electrolytic material dispensed between the ECG electrode surface and the patient's skin. Such dry ECG electrodes 112 can be more comfortable for continuous and/or long term monitoring applications. In various embodiments, the ECG electrodes 112 may be polarizable ECG electrodes 112. Various embodiments may include one or more electrodes 112 formed from conductive polymer coated fibers. Associated description for forming and using electrodes 112 formed from individually conductive polymer coated fibers are described in U.S. provisional patent application No. 63/432,477, which is incorporated herein by reference in its entirety.

In some examples, the therapy electrodes 114 can also be configured to include sensors configured to detect ECG signals as well as other physiological signals of the patient 102. The connection pod 135 can, in some examples, include a signal processor configured to amplify, filter, and digitize these cardiac signals prior to transmitting the cardiac signals to the controller 120. One or more of the therapy electrodes 114 can be configured to deliver one or more therapeutic defibrillating shocks to the body of the patient 102 when the smart garment 110 determines that such treatment is warranted based on the signals detected by the sensing electrodes 112 and processed by the controller 120. Example therapy electrodes 114 can include conductive metal electrodes such as stainless steel electrodes that include, in certain implementations, one or more conductive gel deployment devices configured to deliver conductive gel to the metal electrode prior to delivery of a therapeutic shock.

Some embodiments may be configured to switch between a therapeutic smart garment 110 configuration and a monitoring smart garment 110 configuration that is configured to only monitor a patient 102 (e.g., not provide or perform any therapeutic functions). For example, therapeutic components such as the therapy electrodes 114 and associated circuitry can be optionally decoupled from (or coupled to) or switched out of (or switched in to) the smart garment 110. For example, the smart garment 110 can have therapeutic elements (e.g., defibrillation and/or pacing electrodes, components, and associated circuitry) that are configured to be used when the garment 110 is placed in a therapeutic mode. In examples, the optional therapeutic elements can be physically decoupled from the smart garment 110 as a means to convert the therapeutic smart garment 110 into a monitoring for a specific use (e.g., for operating in a monitoring-only mode) or a patient 102. Alternatively, the therapeutic elements can be deactivated (e.g., by means or a physical or a software switch), essentially rendering the therapeutic smart garment 110 as a monitoring smart garment 110 for a specific physiologic purpose or a particular patient 102. As an example of a software switch, an authorized person can access a protected user interface of the smart garment 110 and select a preconfigured option or perform some other user action via the user interface to deactivate the therapeutic elements of the smart garment 110.

In accordance with one or more examples, the smart garment 110 may provide comfort and functionality under circumstances of human body dynamics, such as bending, twisting, rotation of the upper thorax, semi-reclining, and lying down. These are also positions that a patient may assume if he/she were to become unconscious due to an arrhythmic episode. The design of the garment 110 is generally such that it minimizes bulk, weight, and undesired concentrations of force or pressure while providing the necessary radial forces upon the treatment and sensing electrodes 114, 112 to ensure device functionality. A wearable defibrillator monitor may be disposed in a support holster (not shown) operatively connected to or separate from the smart garment 110. The support holster may be incorporated in a band or belt worn about the patient's waist or thigh.

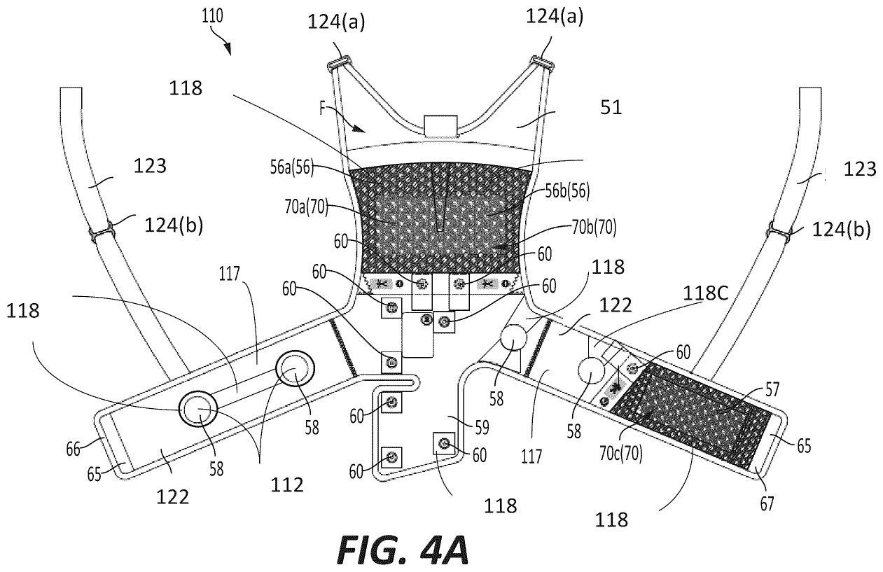

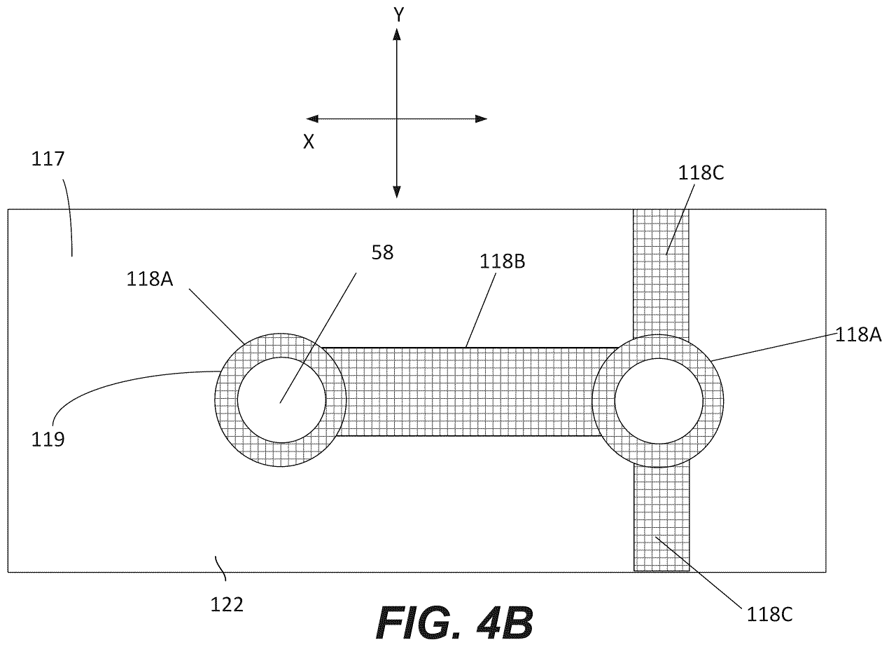

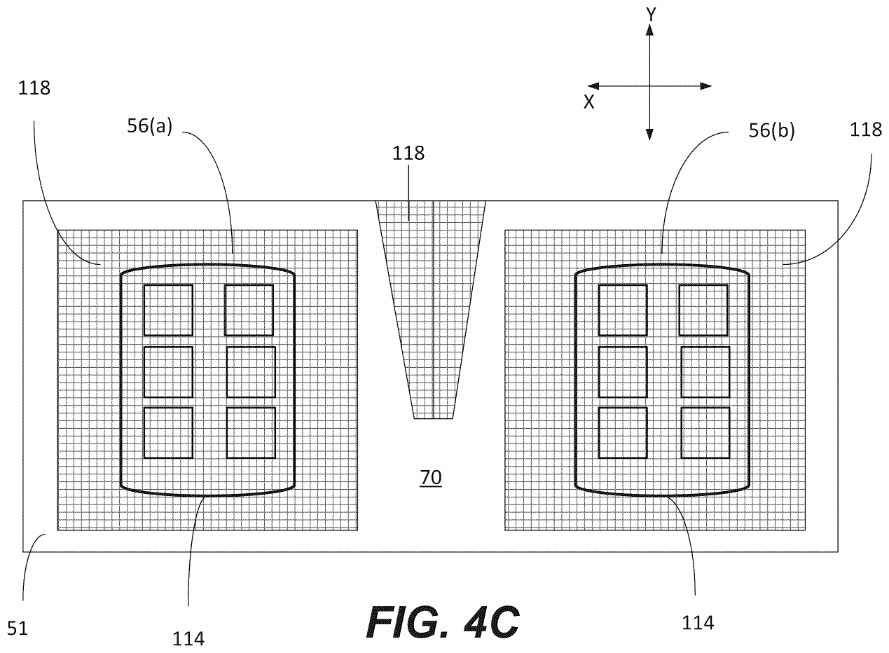

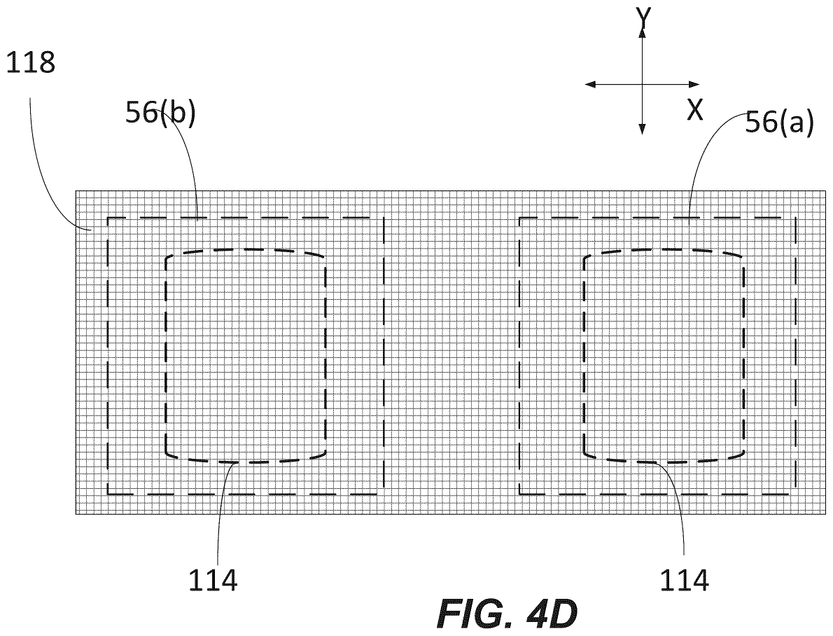

FIG. 4A illustrates an example smart garment 110 according to the present disclosure. The smart garment 110 incorporates additional improvements for enhancing the patient 102 experience wearing the smart garment 110 for an extended period of time. The smart garment 110 examples provided herein promote comfort, aesthetic appearance, coupling between medical device 100 and the patient 102, and/or ease of use or application for older patients 102, or patients 102 with physical infirmities and/or who are physically challenged, including patients 102 with rheumatic conditions, patients with arthritis, and/or patients with autoimmune or inflammatory diseases that affect joints, tendons, ligaments, bones, and muscles of the arm and hand. Patients 102 afflicted with such conditions can properly and/or correctly don the garments 110 described herein.

Features of the smart garments 110 may also help minimize the time needed by patient 102 to assemble, don or remove the smart garment 110. Further, patients 102 benefit from such features, which can facilitate longer wear times, better patient 102 compliance, and improve the reliability of the detected physiological signals and treatment of the patient 102. These features promote ease of use, comfort and an aesthetic appearance for such patient 102 populations. For example, the features include support pockets for the therapeutic electrodes 114 that incorporate rear pocket mesh interfaces 70a and 70b, and a front pocket mesh interface 70c (collectively mesh interface 70) between the therapeutic electrodes 114 and the patient's 102 skin that is more comfortable, less abrasive, and less likely to cause irritation to the patient's 102 skin or a negative reaction.

The patient 102 may be required to wear the smart garment 110 and the components continuously or nearly continuously for extensive periods of time. Over these extensive periods of time, it is desirable to minimize any discomfort while wearing the smart garment as a result of the abrasiveness of the metal materials contained within the interfacing fabric material. As such, patients 102 benefit from a wearable cardioverter defibrillator garment 110 as described herein that includes features for enhancing the patient's 102 experience in wearing the smart garment 110 with respect to wearability and comfort of the garment with respect to the interfacing fabric materials.

These features can encourage patients 102 to wear the smart garment 110 and associated device 100 for longer and/or continuous periods of time with minimal interruptions in the periods of wear. For example, by minimizing interruptions in periods of wear and/or promoting longer wear durations, patients 102 and caregivers can be assured that the smart garment 110 is providing desirable information about as well as protection from adverse cardiac events such as ventricular tachycardia and/or ventricular fibrillation, among others. Moreover, when the patient's 102 wear time and/or compliance is improved, the device 100 can collect information on arrhythmias that are not immediately life-threatening, but may be useful to monitor for the patient's cardiac health. Such arrhythmic conditions can include onset and/or offset of bradycardia, tachycardia, atrial fibrillation, pauses, ectopic beats bigeminy, trigeminy events among others. For instance, episodes of bradycardia, tachycardia, or atrial fibrillation can last several minutes and/or hours.

The smart garments 110 herein provide features that encourage patients 102 to keep the device 100 on for longer and/or uninterrupted periods of time, thereby increasing the quality of data collected about such arrhythmias. Additionally, features as described herein, including, the mesh interfaces for the therapeutic electrode support pockets promotes better patient compliance resulting in lower false positives and noise in the physiological signals collected from ECG electrodes and other sensors disposed within the smart garment. For example, when patients 102 wear the device for longer and/or uninterrupted periods of time, the device 100 tracks cardiac events and distinguishes such events from noise over time.

The improvements incorporated in the smart garment 110 may provide comfort and wearability to the patient by utilizing a mesh interface 70 made from a layer or layers of fabric material incorporating a reduced amount of conductive metal content. Various embodiments may form the entirety, or portions, of the mesh interface 70 from a multiaxially expandable fabric. Accordingly, various embodiments may provide reliable contact between the therapeutic electrodes 114 and the patient 102 for treatment. The fabric material of the mesh interface 70 may additionally, or alternatively, incorporate component materials that have a soft, comfortable feel on the patient's 102 skin and are configured to wick moisture away from the patient's 102 skin. The fabric material of the mesh interface 70 may be less abrasive and more breathable to the patient's 102 skin and less likely to cause irritation to the patient's skin or a negative reaction.

In accordance with one or more examples, the smart garment 110 is provided to keep the electrodes 114, 112 of an electrode assembly 100 associated with a wearable cardiac therapeutic device in place against the patient's 102 body while remaining comfortable to wear. In particular, the electrode assembly 100 may include the plurality of ECG sensing electrodes 112 configured to sense ECG signals regarding a cardiac function of the patient 102 and the plurality of therapy electrodes 114 configured to deliver transcutaneous defibrillation shocks or transcutaneous pacing pulses or other types of therapeutic electrical pulses, to the patient's 102 heart. For example, the pacing pulses comprises current of one or more of the pacing pulses between 0.1 mA and 300 mA.

It is to be appreciated that the smart garment 110 described herein may be utilized in connection with a wearable of any suitable type or configuration. As shown in FIG. 4A, the smart garment 110 may be provided in the form of a vest or harness having a back portion 51 and sides extending around the front of the patient 102 to form the belt 122. In various embodiments, the back portion 51, the belt 122, or a portion thereof may be formed as a multiaxially expandable fabric.

FIG. 4B schematically shows a magnified view of a portion of the smart garment 110 of FIG. 4A. The smart garment 110 may be comprised of an elastic, low spring rate fabric material that stretches appropriately to keep the electrodes 114, 112 in place against the patient's 102 skin and is lightweight and breathable. As shown in FIG. 4B, electrodes 114, 112 can be removably attached, e.g., via hook and loop fasteners or snap connectors, to attachment regions 58. The component materials of the fabric material may be chosen for functionality, comfort, and biocompatibility. The component materials may be configured to wick perspiration from the skin. For example, the fabric material may comprise a tricot fabric, the tricot fabric comprising nylon and spandex materials. The tricot fabric may comprise approximately 65%-90% nylon material, or more particularly 70%-85% nylon material, or more particularly 77% nylon material. These fabrics may be formed as a multiaxially expandable portion 118.

In some embodiments, a first fabric portion 117 (e.g. body of the garment 110) that is a non-multiaxially expandable portion may be formed from the previously referenced fabrics. In some embodiments, the first fabric portion 117, or other non-multiaxially expandable fabric, may form a majority of the garment 110.

In various embodiments, the multiaxially expandable fabric portion 118 may be formed from a blend of cotton, polyester, and/or elastane (e.g., Lycra®). For example, the fabric portion 118 may be about 80% polyester and about 20% elastane. The fabric portion 118 may be substantially homogeneous throughout. However, in some areas, the expandable fabric portion 118 may have changed ratios of materials. For example, the multiaxially expandable fabric portion 118 may have some areas formed by a 90% polyester and about 10% elastane mix (e.g., to reduce the stretchability of the fabric portion 118). However, the multiaxially expandable fabric portions 118 may be formed from other materials known in the art.

In some implementations, the first fabric portion 117 may be coupled with the multiaxially expandable fabric portion 118 at an interface 119 (e.g., a seam, such as a line along which the two fabric portions are sewn together). In examples, the first fabric portion 117 and the multiaxially expandable fabric portion 118 are both knitted portions. For example, such first fabric portion 117 can be manually joined to the multiaxially expandable fabric portion 118 using a flat bed knitting machine. For example, such first fabric portion 117 can be machine joined to the multiaxially expandable fabric portion 118 using a linking machine, e.g., a high speed linking machine. In examples, linking includes seaming and/or attaching pieces of the foregoing fabric portions together after the pieces have been knitted on a flat-bed knitting machine. In examples, a slacker course of loops of yarn can be created on the linking machine, which connects the two pieces of fabric together.

For example, such first fabric portion 117 can be manually joined to the multiaxially expandable fabric portion 118 using one or more cut and sew methods. For examples, such methods include seaming, such as an open seam (e.g., where the seam allowance, the piece of fabric between the edge of the material and the stitches, is visible) or a closed seam method (e.g., incorporates the seam allowance within the seam finish, making the seam allowance invisible). Seams can include plain seams, double-stitched seams, French seams, bound seams, Flat-felled seams, Welt seams, or lapped seams. In examples, a bias tape (e.g., a narrow strip of fabric) can be folded over an exposed seam to secure and hide edges. In examples, a zigzag stitch can be implemented along a raw edge of the seam to secure the edges and prevent fraying. In examples, a faux overlock stitch can be implemented in the seam. In examples, a reinforced straight stitch can be implemented in the seam. In examples, hemming can be implemented in the seam. In examples, depending on the yarn material and/or make up, ultrasonic bonding techniques, including creation and channeling of high frequency vibratory waves that cause a rapid buildup of heat can be used to implement joins. An example of such a device includes the SeamMaster® General Purpose Ultrasonic Sewing Machine from Sonobond Ultrasonics of West Chester, PA, USA. For examples, fabric materials suited for ultrasonic joins includes thermoplastic fabric and/or film materials including acrylic, nylon, polyester, polyethylene, polypropylene, polyvinylchloride and urethane. Accordingly, one or both of first fabric portion 117 and multiaxially expandable fabric portion 118 comprises materials including thermoplastic fabric and/or film materials including acrylic, nylon, polyester, polyethylene, polypropylene, polyvinylchloride and urethane. In some embodiments, the first fabric portion 117 may be interwoven, adhered, glued, sewn, chemically welded, and/or heat welded with the multiaxially expandable fabric portion 118 at the interface 119

In various embodiments, the first fabric portion 117 and multiaxially expandable fabric portion 118 are integrated into the knitting, e.g., incorporated into a knitting machine software program such that the knitted resulting fabric transitions from multiaxially expandable fabric to non-multiaxially expandable fabric zones in the same fabric material. In this regard, first fabric portion 117 and multiaxially expandable fabric portion 118 are implemented on a common fabric. For example, a computer-based knitted machine from, e.g., Stoll of the Karl Mayer Group based and based in Dayton, OH, USA, can be used to knit such materials as described herein. In this regard, as a first step, a knitting program comprising computer-readable code executable by a computer coupled to the knitting machine can be implemented to cause the fabric to knit a first non-multiaxially expandable fabric zone in a first, predetermined region of the fabric. As a second step, the computer-readable code can instruct the knitting machine to knit multiaxially expandable fabric zones in a second predetermined region of the fabric (and in any other regions). In regions of the common fabric where it is desirable to implement the multiaxially expandable fabric portion 118, techniques for developing the multiaxially expandable fabric portion 118 as described herein can be implemented through the knitting program. In such methods, regions of first fabric portion 117 and regions of multiaxially expandable fabric portion 118 are disposed on a same fabric, and as such the different regions result from use of different knitting techniques to create the desired regions.

The multiaxially expandable fabric portion 118 may be made of the same or similar materials as the first fabric portion 117. However, as discussed below, the multiaxially expandable fabric portion 118 may be formed so as to be multiaxially expandable. In some other embodiments, the first fabric portion 117 and the multiaxially expandable portion 118 may be formed of different materials. As shown in FIG. 4A, the smart garment 110 may be provided in the form of a vest or harness having a back portion 51 and sides extending around the front of the patient 102 to form the belt 122. In various embodiments, the back portion 51, the belt 122, or a portion thereof may be formed as a multiaxially expandable fabric. In some embodiments, the multiaxially expandable fabric portion 118 may be positioned adjacent to the electrodes 114, 112 (e.g., surrounding and/or extending from the electrodes) to inhibit electrode flipping, rotation, twisting, or other undesirable movement, as patients 102 with torsos of various shapes and/or sizes perform their normal activity throughout the course of the day. It is recognized that multiaxially expandable can refer to expansion of the knit/weave structure of the portion 118 simultaneously in two or more directions (e.g. X, Y and/or Z directions). For example, if the knit/woven material of the portion 118 is subjected to stretch/expansion in the X direction due to movement of the wearer, the knit/woven material of the portion 118 would as a result also expand simultaneously in the Y direction. For example, if the knit/woven material of the portion 118 is subjected to stretch/expansion in the X direction due to movement of the wearer, the knit/woven material of the portion 118 would as a result also expand simultaneously in the Z direction. For example, if the knit/woven material of the portion 118 is subjected to stretch/expansion in the Y direction due to movement of the wearer, the knit/woven material of the portion 118 would as a result also expand simultaneously in the Z direction. For example, if the knit/woven material of the portion 118 is subjected to stretch/expansion in the X and Y directions due to movement of the wearer, the knit/woven material of the portion 118 would as a result also expand simultaneously in the Z direction.

To that end, in various embodiments an ECG electrode 112 may be enveloped by the multiaxially expandable fabric portion 118. In some embodiments, the multiaxially expandable fabric portion 118 and the first fabric portion 117 are interwoven or overlayed adjacent to an ECG electrode 112 and/or therapy electrodes 114. In some embodiments, the expandable fabric portion 118 is configured to contact the electrode 112 directly. The expandable fabric portion 118 is then coupled with the remainder of the garment 110 by directly coupling with the first fabric portion 117.

In various embodiments, the electrode attachment points 58 may be surrounded by multiaxially expandable fabric section 118A. As discussed further below, the multiaxially expandable fabric section 118A may also be multiaxially contractible. Thus, when the multiaxially expandable fabric section 118A is compressed in a first direction (e.g., by contact with the patient's 102 skin) it may contract in a second perpendicular direction. For example, when the sections 118A-118C are stretched in the X-direction (e.g., circumferentially about the torso of the patient in an anatomical axial plane), the respective section 118A-118C may expand in the Y-direction and/or in the Z-direction (e.g., towards the patient 102), or vice-versa. Similarly, when the sections 118A-118C are compressed in the X-direction (e.g., circumferentially about the torso of the patient in an anatomical axial plane), the respective section 118A-118C may contract in the Y-direction and/or in the Z-direction (e.g., towards the patient 102), or vice-versa. Configuring the fabric portion 118 to expand or contract in a particular direction provides a number of advantages discussed further below. It is recognized that multiaxially expandable can refer to expansion of the knit/weave structure of the portion 118 simultaneously in two or more directions (e.g. X, Y and/or Z directions). For example, if the knit/woven material of the portion 118 is subjected to stretch/expansion in the X direction due to movement of the wearer, the knit/woven material of the portion 118 would as a result also expand simultaneously in the Y direction. For example, if the knit/woven material of the portion 118 is subjected to stretch/expansion in the X direction due to movement of the wearer, the knit/woven material of the portion 118 would as a result also expand simultaneously in the Z direction. For example, if the knit/woven material of the portion 118 is subjected to stretch/expansion in the Y direction due to movement of the wearer, the knit/woven material of the portion 118 would as a result also expand simultaneously in the Z direction. For example, if the knit/woven material of the portion 118 is subjected to stretch/expansion in the X and Y directions due to movement of the wearer, the knit/woven material of the portion 118 would as a result also expand simultaneously in the Z direction.

The smart garment is configured to maintain the electrical contact between the one or more of the plurality of ECG electrodes 112 and skin of the patient at least by pressing the one or more of the plurality of ECG electrodes against the skin of the patient of the smart garment at a predetermined range of pressures. In embodiments, the multiaxially expandable fabric portion 118A incorporated into garment 110 may be configured to cause the garment 110 to maintain a contact pressure between the patient's 102 skin and an ECG electrode 112 an ECG electrode 112 of between about 5 and about 150 mm Hg (e.g., 75 mm Hg). In embodiments, the multiaxially expandable fabric portion 118A incorporated into garment 110 may be configured to cause the garment 110 to maintain a contact pressure between the patient's 102 skin and an ECG electrode 112 of between about 5 and about 150 mm Hg (e.g., 50 mm Hg). In embodiments, the multiaxially expandable fabric portion 118A incorporated into garment 110 may be configured to cause the garment 110 to maintain a contact pressure between the patient's 102 skin and an ECG electrode 112 of between about 5 and about 50 mm Hg (e.g., 25 mm Hg). In embodiments, the multiaxially expandable fabric portion 118A incorporated into garment 110 may be configured to cause the garment 110 to maintain a contact pressure between the patient's 102 skin and an ECG electrode 112 of between about 5 and about 40 mm Hg (e.g., 15 mm Hg). In embodiments, the multiaxially expandable fabric portion 118A incorporated into garment 110 may be configured to cause the garment 110 to maintain a contact pressure between the patient's 102 skin and an ECG electrode 112 of between about 0.1 psi and about 0.8 psi (e.g., 0.6 psi). In embodiments, the multiaxially expandable fabric portion 118A incorporated into garment 110 may be configured to cause the garment 110 to maintain a contact pressure between the patient's 102 skin and an ECG electrode 112 of between about 0.1 psi and about 2 psi (e.g., 1 psi). In embodiments, the multiaxially expandable fabric portion 118A incorporated into garment 110 may be configured to cause the garment 110 to maintain a contact pressure between the patient's 102 skin and an ECG electrode 112 of between about 0.1 psi and about 3 psi (e.g., 1.5 psi).

The smart garment is configured to maintain the electrical contact between the one or more of the plurality of therapy electrodes 114 (114a, 114b, and 114c) and skin of the patient at least by pressing the one or more of the plurality of ECG electrodes 112 against the skin of the patient 102 of the smart garment 110 at a predetermined range of pressures. In embodiments, the multiaxially expandable fabric portion 118A incorporated into garment 110 may be configured to cause the garment 110 to maintain a contact pressure between the patient's 102 skin and a therapy electrode 114 of between about 5 and about 150 mm Hg (e.g., 75 mm Hg). In embodiments, the multiaxially expandable fabric portion 118A incorporated into garment 110 may be configured to cause the garment 110 to maintain a contact pressure between the patient's 102 skin and a therapy electrode 114 of between about 5 and about 150 mm Hg (e.g., 50 mm Hg). In embodiments, the multiaxially expandable fabric portion 118A incorporated into garment 110 may be configured to cause the garment 110 to maintain a contact pressure between the patient's 102 skin and a therapy electrode 114 of between about 5 and about 50 mm Hg (e.g., 25 mm Hg). In embodiments, the multiaxially expandable fabric portion 118A incorporated into garment 110 may be configured to cause the garment 110 to maintain a contact pressure between the patient's 102 skin and a therapy electrode 114 of between about 5 and about 40 mm Hg (e.g., 15 mm Hg). In embodiments, the multiaxially expandable fabric portion 118A incorporated into garment 110 may be configured to cause the garment 110 to maintain a contact pressure between the patient's 102 skin and a therapy electrode 114 of between about 0.1 psi and about 0.8 psi (e.g., 0.6 psi). In embodiments, the multiaxially expandable fabric portion 118A incorporated into garment 110 may be configured to cause the garment 110 to maintain a contact pressure between the patient's 102 skin and a therapy electrode 114 of between about 0.1 psi and about 2 psi (e.g., 1 psi). In embodiments, the multiaxially expandable fabric portion 118A incorporated into garment 110 may be configured to cause the garment 110 to maintain a contact pressure between the patient's 102 skin and a therapy electrode 114 of between about 0.1 psi and about 3 psi (e.g., 1.5 psi).

The smart garment 110 may include multiple interconnected sections 118A, 118B, and/or 118C. For example, the multiaxially expandable fabric sections 118A surrounding the electrode attachment points 58 may include multiaxially expandable fabric 118B extending therebetween. While physically coupled, the various sections 118A and 118B may or may not be expandably isolated from one another. That is, expansion or contraction of one section 118A may not necessarily cause another section 118B to similarly contract or expand. However, in some embodiments, expansion and contraction from one section 118A may cause another section 118B to experience a similar expansion or contraction. Coupling various multiaxially expandable sections 118A using an extended multiaxially expandable fabric section 118B may assist with achieving a desired electrode 112 contact pressure with the patient 102. Additionally, or alternatively, the extended multiaxially expandable fabric section 118B may help retain the electrodes 112 in their proper orientation relative to the patient 102 when the garment 110 is stretched during use. To that end, additional multiaxially expandable fabric section 118C may be positioned to help properly maintain electrodes 112 in the appropriate orientation. For example, expandable material section 118C may run transversely from the electrode attachment point 58 towards an edge of the garment 110 (e.g., towards an edge of the belt 122).

The various sections 118A, 118B, and/or 118C of the multiaxially expandable fabric portion 118 may overlap one another. Additionally, or alternatively, the various sections of multiaxially expandable fabric portion 118 material may overlap with the first fabric portion 117. Any of the multiaxially expandable fabric sections 118A, 118B, and/or 118C may be formed as a double layer.

Returning to FIG. 4A, the ends 66, 67 of the belt 122 may be connected at the front of the patient 102 by a closure mechanism 65. The smart garment 110 may further include two straps 123 connecting the back portion 51 to the belt 122 at the front of the patient 102. In examples, one or more devices 100 as described herein can be disposed on one or both straps 123. For example, the devices 100 may be permanently coupled to one or both straps 123. For example, the devices may be removably coupled to one or both straps 123. In examples, one or more ECG electrodes 112 as described herein can be disposed on one or both straps 123. For example, the ECG electrodes 112 may be permanently coupled to one or both straps 123. For example, the ECG electrodes 112 may be removably coupled to one or both straps 123. The straps 123 have an adjustable size to provide a more customized fit to the patient 102. In some embodiments, the straps 123 may be include a multiaxially expandable fabric portion 118. In locating such multiaxially expandable fabric portion 118 in the strap 123, the smart garment 110 is configured to cause the overall garment structure to push devices 100 disposed in the smart garment (e.g., on one or both straps 123 or elsewhere on smart garment 110) towards the body of the patient when the strap 123 is stretched along its length during wear. For example, multiaxially expandable fabric portion 118 in the strap 123 of smart garment 110 can cause the smart garment 110 to exert forces against the ECG and therapy electrodes disposed within the garment 110. Such forces can cause the ECG and therapy electrodes to maintain proper contact with the patient's skin during use of the smart garment 110. First strap slides 124a may be provided to connect the straps 123 to the back portion 51 of the smart garment. Second strap slides 124b may be provided along the straps 123 to facilitate size adjustment of the straps 123. The straps 123 with such multiaxially expandable fabric portion 118 may be removably attached to the belt 122 at the front of the patient 102. In implementations, the straps 123 with such multiaxially expandable fabric portion 118 may be permanently secured to the belt 122 at the front of the patient 102, such that the strap 123 cannot be separated from the belt 122 without destruction of the smart garment 110.

The smart garment 110 may be configured for one-sided assembly of the electrode assembly 100 onto the smart garment 110 such that the smart garment 110 does not need to be flipped or turned over in order to properly position the therapy electrodes 114 and the sensing electrodes 112 on the smart garment 110. The inside surface of the back portion 51 of the smart garment 110 includes one or more pocket(s) 56b, 56c (together 56) for receiving one or two therapy electrodes 114 to hold the electrode(s) 114 in position against the patient's 102 back. The one or more pockets 56 includes a mesh interface 70 (or mesh interfaces 70a, 70b) incorporating a plurality of conductive fibers for transmitting electrical energy from the therapy electrode towards the patient's skin. In examples, the one or more pockets 56 can be configured to include dielectric (i.e., non-conductive) fibers comprising at least one nonmetallic material and a plurality of conductive fibers or particles therein, as well as a plurality of openings defined therein. The garment region forming the rear of the pockets 56 (e.g., the fabric opposing the mesh portion 70) may include a multiaxially expandable portion 118 configured to push the therapy electrode towards the patient's skin when the garment region is stretched along traverse orientations (e.g., orientations substantially perpendicular to the axis oriented towards the patient).

Alternatively or additionally, in some embodiments, one or more pockets as described above for receiving the therapy electrodes 114 may be accessible from an outside surface of the smart garment 110 (e.g., the surface of the garment that is facing away from skin of the patient) rather than an inside surface of the garment. In such implementation, the one or more pockets includes a mesh interface (similar to mesh interface 70 or mesh interfaces 70a, 70b as described above) incorporating a plurality of non-conductive fibers comprising at least one nonmetallic material and a plurality of conductive fibers or particles therein, as well as a plurality of openings defined therein. The garment region forming the rear of the pockets 56 may include a multiaxially expandable portion 118 configured to push the therapy electrode towards the patient's skin when the garment region is stretched along traverse orientations.

The mesh interface 70 is configured to physically separate the metallic therapy electrode(s) 114 from the skin of the patient 102 while allowing a conductive gel that may be automatically extruded from a plurality of holes in the electrode(s) 114 to easily pass through to the skin of the patient 102. The forces applied to the electrode(s) 114 by the mesh interface 70, in addition to the use of the conductive gel, may help ensure that proper contact and electrical conductivity with the patient's 102 body are maintained, even during body motions. The mesh interface 70 also maintains electrical contact between the electrode(s) 114 through the material of the mesh interface 70 before the conductive gel is dispensed, which allows for monitoring of the therapy electrode(s) 114 to ensure that the electrode(s) 114 are positioned against the skin such that a warning may be provided by the smart garment 110 if the therapy electrode(s) 114 is not properly positioned. Another pocket, front pocket 57 including a mesh interface 70c according to the same construction is included on an inside surface of the belt 122 for receiving a front therapy electrode 114c and holding the electrode 114 in position against the patient's 102 left side. Similar to the configuration of the rear pockets, the garment region forming the rear of the front pocket 57 may include a multiaxially expandable portion 118 configured to push the front therapy electrode towards the patient's skin when the garment region is stretched along a circumferential orientation (e.g., in a direction along the circumference of torso of the patient in an anatomical axial plane). In various embodiments, the mesh interface 70 of any of the pockets 56, 57 may be formed as a multiaxially expandable fabric portion 118. Accordingly, the openings of the mesh may be configured to expand as the belt 122, back portion 51, and/or pockets 56, 57 are stretched by the patient's 102 body.

After assembly of the therapy electrode(s) 114 into the respective pocket(s) 56, 57, the pocket(s) 56, 57 are closed on the smart garment 110, by a fastener or fasteners 60, such as a button or snap. Two rear pockets 56B and 56C, and one front pocket 57 are shown corresponding to the two rear therapy electrodes 114a and 114b, and front therapy electrode 114c. In other implementations, fewer or more rear or front pockets and/or therapy electrodes may be provided. For example, the garment 110 can include two rear pockets and two front pockets, these pockets configured to receive two rear therapy electrodes and two front therapy electrodes. For example, the garment 110 can include three rear pockets and three front pockets, these pockets configured to receive three rear therapy electrodes and three front therapy electrodes. In such examples, the rear or front pockets can include corresponding mesh interface as described herein. All of the pockets may include a multiaxially expandable portion.

The back portion 51 and the belt 122 of the smart garment 110 may further incorporate attachment points 58 for supporting the sensing electrodes 112 in positions against the patient's 102 skin in spaced locations around the circumference of the patient's 102 chest. The attachment points 58 may include hook-and-loop fasteners for attaching ECG sensing electrodes 112 having a corresponding fastener disposed thereon to the inside surface of the belt 122. The attachment points 58 may be color coded to provide guidance for appropriately connecting the sensing electrodes 112 to the smart garment 110. Additionally or alternatively, one or more of the ECG sensing electrodes can be permanently integrated into the belt 122 of the smart garment 110, e.g., such that they cannot be removed/replaced by a patient during use. The smart garment 110 may further be provided with a flap 59 extending from the back portion 51.

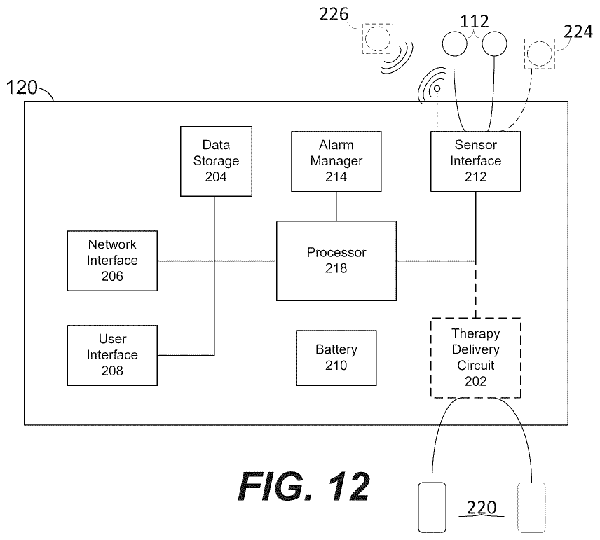

The flap 59 and the back portion 51 include fasteners 60 for connecting the flap 59 to the inside surface of the back portion 51 to define a pouch or pocket for holding a connection pod 135. The connection pod 135 may include processing and/or vibrational circuitry of the electrode assembly 100. For example, the connection pod 135 can include ECG acquisition and conditioning circuitry configured to receive ECG signals from the plurality of ECG sensing electrodes 112, amplify the signals, condition (e.g., using filter circuits) to remove noise, and sample the signal to produce a digitized ECG signal corresponding to the analog ECG input. In examples, the connection pod 135 can also include vibrational circuitry configured to receive an input from a controller (e.g., controller 120 shown in FIG. 12 below) and provide the patient 102 a vibrational alarm or notification as appropriate. The outer surface of the belt 122 may incorporate a schematic imprinted on the fabric for assisting the patient or medical professional in assembling the electrode assembly 100 onto the smart garment 110. Portions of the garment 110 of the flap 59, and/or portions adjacent to the fasteners 60 and the attachment points 58 may also be formed as a multiaxially expandable fabric. In examples, the garment fabric in the region around the location of the connection pod 135 can include multiaxially expandable fabric configured such that the connection pod 135 is caused to push against the body of the patient when the multiaxially expandable fabric in the region is subject to transverse forces (e.g., stretch forces in directions that are substantially perpendicular to an axis oriented towards the patient's skin).

FIG. 4C schematically shows a magnified view of another portion of the smart garment 110 of FIG. 4A. In particular, a front view of the pockets 56 that hold the therapy electrodes 114 are shown. In FIG. 4C, the therapy electrodes 114 are shown positioned within pockets 56(a) and 56(b). The therapy electrodes 114 are visible through, for example, the mesh 70. As is discussed in greater detail further below, the pockets 56 and/or other portions of the garment 110 adjacent to the therapy electrodes 114 may be formed of multiaxially expandable material portions 118. For example, a part of the back 51 of the garment between the pockets 56 may be formed as a multiaxially expandable fabric portion 118 and configured to cause the garment 110 to push the therapy electrodes towards the skin of the patient when the back 51 portion of the garment is subject to transverse forces (e.g., stretch forces in directions that are substantially perpendicular to an axis oriented towards the patient's skin).

FIG. 4D schematically shows a rear view of the back 51 of the portion of the smart garment 110 of FIG. 4C. The pockets 56 and electrodes 114, which are not visible from the rear view are shown in dashed lined. In various embodiments, the entirety of the rear of the back 51 of the smart garment 110 may be formed as a multiaxially expandable material portion 118.

In various embodiments, the pockets 56 may be surrounded and/or formed as multiaxially expandable fabric portion 118. Thus, when the multiaxially expandable fabric portion 118 is compressed in the first direction (e.g., by contact with the patient's 102 skin) it may contract in a second perpendicular direction. For example, when the multiaxially expandable portion 118 is stretched in the X-direction (e.g., circumferentially), it may also expand in the Y-direction and/or in the Z-direction (e.g., towards the patient 102), or vice-versa.

Configuring the fabric portion 118 to expand or contract in a particular direction provides a number of advantages. For example, the fabric portion 118 may be configured to expand in the Z-direction (e.g., inwardly towards the patient 102) when the garment 110 is stretched circumferentially so as to keep the ECG electrodes 112 or therapy electrodes 114 tighter to the skin. Additionally, or alternatively, the garment 110 may be configured to expand in the Y-direction (e.g., up and down relative to the patient 102) when the garment 110 is stretched circumferentially so as to keep the ECG electrodes 112 or therapy electrodes 114 in a desired position relative to the patient 102.

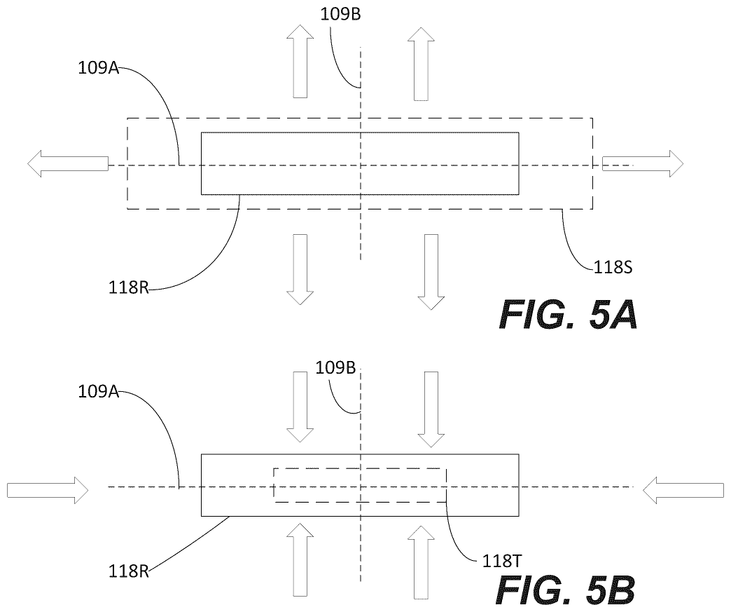

FIG. 5A schematically shows a multiaxially (e.g., biaxially) expandable fabric portion 118 in accordance with illustrative embodiments of the invention in this disclosure. The biaxially expandable fabric portion 118 has a resting configuration 118R, at which it is at rest when no external forces are applied to the fabric portion 118. When the fabric 118 is stretched along a first axis 109A, the fabric 118 also experiences a stretch along a second perpendicular axis 109B, as shown in the stretched configuration 118S. In this example, a longitudinal stretch (i.e., along longitudinal axis 109A) causes a corresponding stretch along the transverse axis 109B. It should be apparent that the fabric portion 118 is configured to also expand along the longitudinal axis 109A as it is stretched along the transverse axis 109B. Indeed, discussion of the biaxial expansion as occurring along a longitudinal and transverse axes 109A, 109B is for discussion purposes only. It should be apparent that various embodiments may simultaneously expand along any two perpendicular axes (including a Z-axis, not shown). In this way, the biaxially expandable fabric portion 118 has a negative Poisson's ratio. Examples of biaxially expandable fabric portions 118 include auxetic fabrics.

FIG. 5B schematically shows the biaxially expandable fabric portion 118 of FIG. 5A in a compressed configuration. In some embodiments, as the biaxially expandable fabric portion 118 is compressed along the first axis 109A, the fabric portion 118 contracts along a second perpendicular axis 109B. For example, the fabric portion 118 may be compressed from its relaxed configuration 118R inwardly in a longitudinal direction (e.g., as shown by arrows along the longitudinal axis 109A). As the fabric portion 118 is compressed in the first direction, it also contracts along the second perpendicular direction, as shown in the compressed configuration 118T. Discussion of the biaxial contraction as occurring along a longitudinal and transverse axes 109A, 109B is for discussion purposes only. It should be apparent that various embodiments may contract along any two perpendicular axis (including a Z-axis, not shown). In this way, the biaxially expandable fabric has a negative Poisson's ratio.

Thus, in various embodiments, the biaxially expandable fabric portion 118 expands along two perpendicular directions when a tension force is applied to the fabric. However, the biaxially expandable fabric portion 118 contracts along the two perpendicular directions when a compression force is applied to the fabric portion 118.

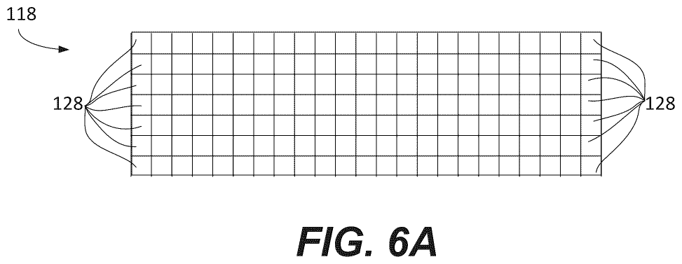

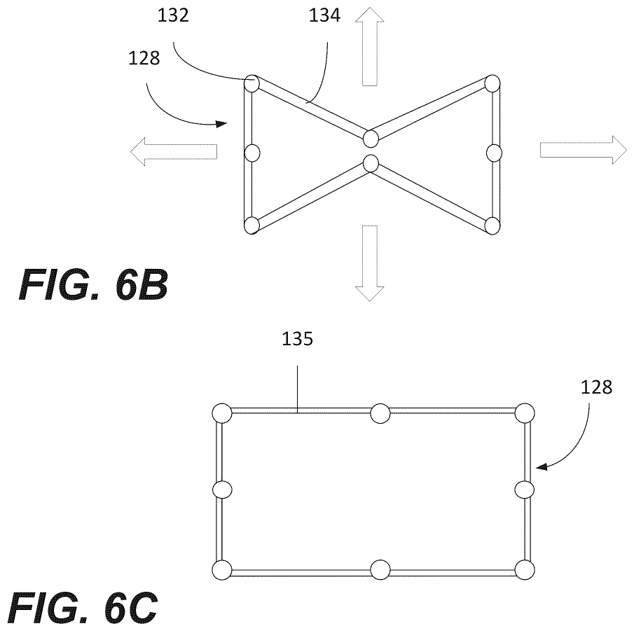

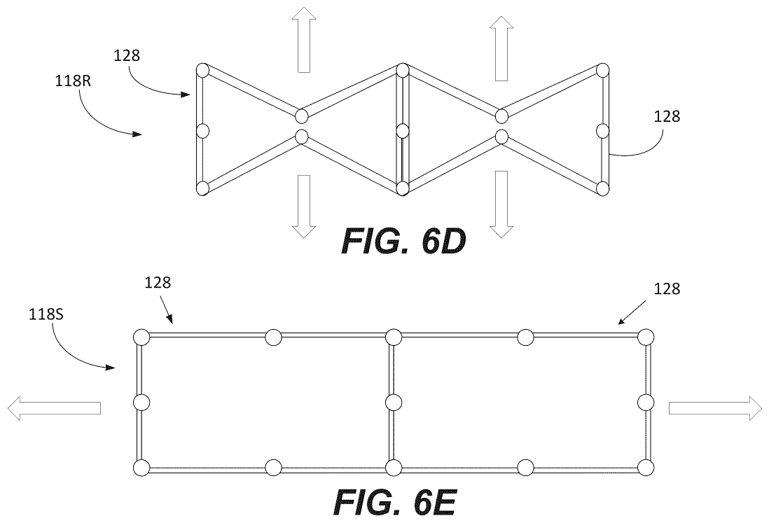

FIG. 6A schematically shows details of an example biaxially expandable fabric portion 118 in accordance with illustrative embodiments in this disclosure. In various embodiments, the biaxially expandable fabric portion 118 is formed from one or more materials (e.g., that are substantially similar to the first fabric portion 117) having an expandable cell 128. FIGS. 6B-6E schematically show details of an embodiment of the biaxially expandable fabric portion 118 of FIG. 6A.

FIGS. 6B and 6C schematically show a single cell 128 of the biaxially expandable fabric portion 118 in accordance with illustrative embodiments. The biaxially expandable fabric portion 118 may be formed of a plurality of individual cells 128 movably coupled together. FIG. 6B schematically shows the single cell 128 when the biaxially expandable fabric portion 118 is in the resting configuration. FIG. 6C schematically shows the single cell 128 when the biaxially expandable fabric portion 118 is in the stretched configuration. As can be seen, the cell 128 may contain pivotable points (referred to as joints 132) around which structures 134 of the cell 128 (e.g., fabric) may pivot. In example implementations, some sub-components of the cell 128 (e.g., joints 132 and/or structures 134) may be formed from positive Poisson's ratio materials. In such implementations, the overall structure of cell 128 can exhibit a negative Poisson's ratio property. For example, it can be seen that the length and width of the cell 128 on FIG. 6B increases in the stretched configuration shown in FIG. 6C. However, the structures 134 themselves may have a decreased width relative to their increased length (e.g., as a normally positive Poisson's ratio stretched material behaves). It is recognized that the structures 134 can be assembled using knit/woven techniques and thus comprise fabric material, e.g. yarns or threads such as nonconductive materials. It is recognized that the structures 134 can be assembled using knit/woven techniques and thus comprise metallic material, e.g. metallic yarns or threads as conductive materials. It is recognized that the structures 134 can be assembled using knit/woven techniques and thus comprise both metallic and fabric material, e.g. yarns or threads including both nonconductive and conductive materials. As mentioned previously, the multiaxially expandable fabric portions may be multiaxially expandable and multiaxially contractible. Accordingly, the cells 128 of the fabric portion 118 may also be multiaxially contractible. Thus, when the cell structure of FIG. 6E is contracted, the cells may return to the structure shown in FIG. 6D.

FIGS. 6D and 6E schematically show the biaxially expandable fabric portion 118 in accordance with illustrative embodiments. In particular, FIG. 6D shows the biaxially expandable fabric portion 118 in the resting configuration 118R. FIG. 6E shows the biaxially expandable fabric portion 118 in the stretched configuration 118S.

For simplicity, the cells 128 of FIGS. 6B-6C are shown to describe the behavior of the biaxially expandable fabric portion 118. However, it should be understood that various embodiments are not limited to the shape of cells 128 shown here. Indeed, many different cell 128 shapes may be used. For example, each of the cells 128 may have an inward concave honeycomb shape. Furthermore, some embodiments may use structures 134 having a negative Poisson's ratio. It should also be understood that a variety of different materials and methods may be used to form the cells 128.

As the fabric 118R is stretched in the first direction, the fabric 118R also stretches along the second perpendicular direction, as shown in the stretched configuration 118S. In this example, a transverse stretch (represented by the arrows in FIG. 6D) causes a corresponding longitudinal stretch (represented by the arrows in FIG. 6E).

In various embodiments, an example multiaxially expandable fabric portion 118 as described herein may be implemented by multiaxially expandable fiber, multiaxially expandable yarn, or both a multiaxially expandable fiber and a multiaxially expandable fabric yarn. For example, the multiaxially expandable yarn may include a double helix yarn. The double helix yarn has a core material and a wrap material. The wrap material may include an ultra-high molecular weight polyethylene fiber and a polyurethane core material. Some embodiments may include a fiber-based non-conductive yarn having a multiaxially expandable fabric structural unit. The fiber-based yarn may have a reentrant hexagonal structural unit when in the unstretched configuration 118R, and a honeycomb structural unit in a stretched configuration 118S. The fiber-based yarn may be lengthened between 5% and 25% in the stretched position relative to the unstretched position.

Among other ways, the multiaxially expandable fabric portion 118 may be formed using a 3D printing process, including an additive printing process. Alternatively, the multiaxially expandable fabric portion may be a woven fabric portion and/or a knitted fabric portion. The knitted fabric portion may be formed using a knitting machine, such as a flat-bed knitting machine or a V-bed knitting machine. Some embodiments may also form the multiaxially expandable fabric portion 118 using a photolithography process.

It should be apparent that in various embodiments the multiaxially expandable material may be configured to expand in more than two perpendicular directions. For example, the material may be triaxially expandable, i.e., configured to expand along three perpendicular axes. In various embodiments, these types of materials may generically be referred to as multiaxially expandable materials.

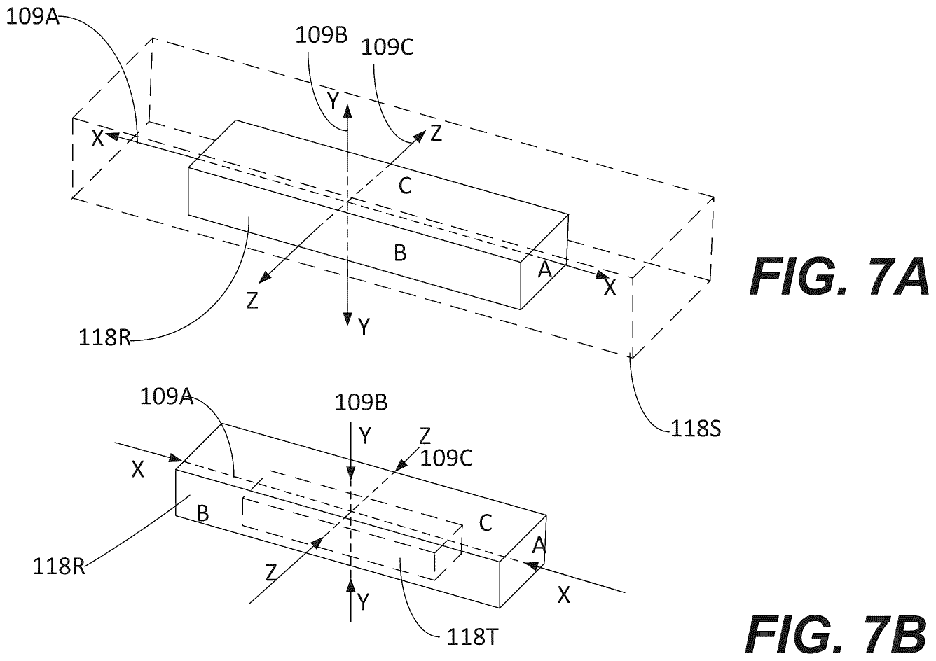

For example, FIG. 7A schematically shows a triaxially expandable fabric portion in accordance with illustrative embodiments in this disclosure. The triaxially expandable fabric portion 118 has a resting configuration 118R when no external forces are applied to the fabric portion 118. As shown in the stretched configuration 118S, the fabric 118 stretches along the second perpendicular axis 109B (e.g., the Y-axis) as it stretches along the first axis 109A (e.g., the X-axis. In this example, a longitudinal stretch (i.e., along X-axis 109A) causes a corresponding stretch along the transverse axis 109B (i.e., along Y-axis 109C). Furthermore, the fabric 118 also stretches along a third perpendicular axis 109C (i.e., along the Z-axis). It should be apparent that the fabric portion 118 also expands along the X-axis 109A and the Z-axis 109C as it is stretched along the Y-axis 109B. In a similar manner, the fabric portion 118 expands along the X-axis 109A and the Y-axis 109B as it is stretched along the Z-axis 109C. In this way, the triaxially or other multiaxially expandable fabric portion 118 has a negative Poisson's ratio. Examples of multiaxial expandable fabric portions 118 include auxetic fabrics.

FIG. 7B schematically shows the triaxially expandable fabric portion 118 of FIG. 7A in a compressed configuration 118T. In some embodiments, as the triaxially expandable fabric portion 118 is compressed along the first axis 109A, the fabric portion 118 contracts along the second perpendicular axis 109B and the third perpendicular axis 109C. For example, the fabric portion 118 may be compressed from its relaxed configuration 118R inwardly in a longitudinal direction (e.g., as shown by arrows along the longitudinal axis 109A). As the fabric portion 118 is compressed in a first direction, it also contracts along the second perpendicular direction 109B and the third perpendicular direction 109C, as shown in the compressed configuration 118T. Discussion of the triaxial contraction as occurring along a longitudinal and transverse axes 109A, 109B is for discussion purposes only. It should be apparent that various embodiments may contract along any three or more perpendicular axis. In this way, the triaxially expandable fabric portion 118 has a negative Poisson's ratio.

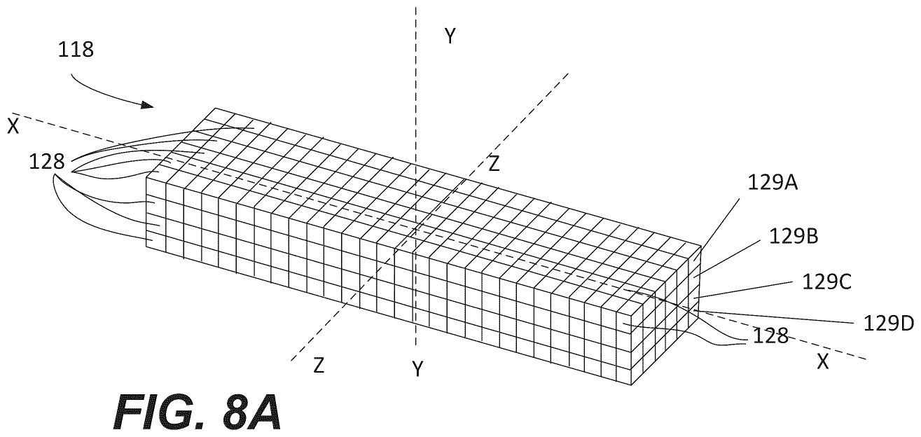

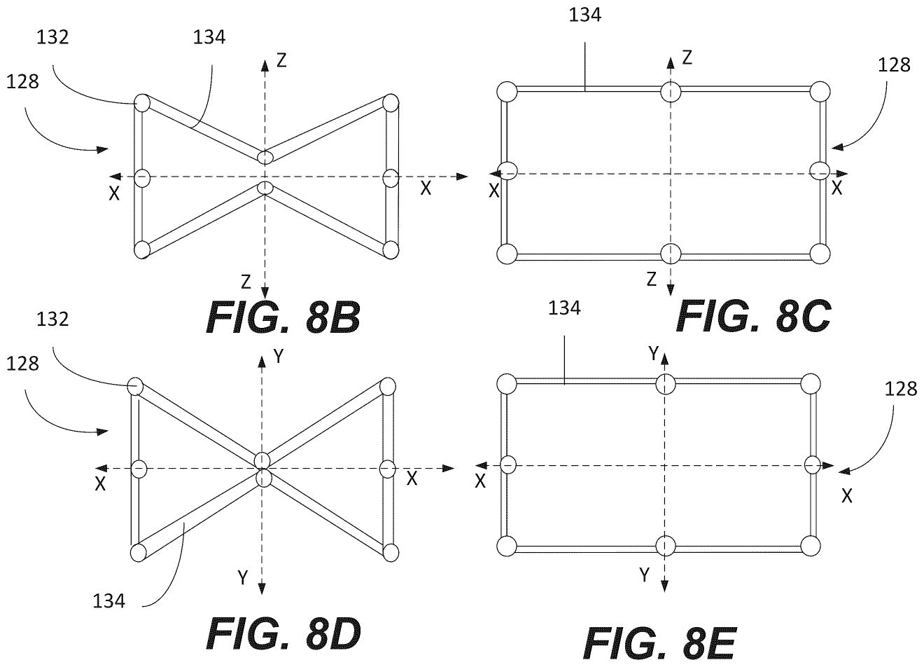

FIG. 8A schematically shows details of the triaxially expandable fabric portion 118 in accordance with illustrative embodiments in this disclosure. In various embodiments, the triaxially expandable fabric portion 118 is formed from one or more materials (e.g., that may be substantially similar to the first portion 117) having an expandable cell 128. FIGS. 8B-8E schematically show details of an embodiment of the multiaxially expandable fabric portion of FIG. 8A. In various embodiments, the multiaxially expandable fabric portion 118 may be expandable along at least three perpendicular axes.

The fabric portion 118 shown in FIG. 8A is considered a multi-cellular-layer fabric portion 118. In particular the fabric portion 118 may be considered a four-cellular-layer fabric portion 118, defined by the smallest numbers of cellular layers 129 in any given axis of the fabric portion 118. In this example, there are four cellular layers 129A-129D along the Y-axis, while the Z-axis and X-axis have considerably more cellular layers 129. Various embodiments may form fabric with between 1 and 100 cellular layers 129. For example, some embodiments could include a single cellular layer 129, double cellular layer 129, triple cellular layer 129. Furthermore, it should be understood that shapes of cellular layers 129 are not limited to rectangular or cubical shapes. These shapes are provided merely to facilitate discussion of illustrative embodiments. Various embodiments may have any shape with expandable properties in multiple different perpendicular axes. Additionally, the individual cells 128 shown represented by small cubes are merely for discussion purposes, and not intended to limit various embodiments.

FIGS. 8B and 8D schematically show different views of the cell 128 of the triaxially expandable fabric portion 118 in accordance with illustrative embodiments. The triaxially expandable fabric portion 118 may be formed of a plurality of the individual cells 128 coupled together. FIG. 8B schematically shows a top view of the single cell 128 when the triaxially expandable fabric portion 118 is in the resting configuration 118R. FIG. 8C schematically shows the top view of the single cell 128 when the triaxially expandable fabric portion 118 is in the stretched configuration 118S. As can be seen, the cell 128 may contain pivotable points (referred to as joints 132) around which structures 134 of the cell 128 (e.g., fabric) may pivot.

FIG. 8D schematically shows a side view of the cell 128 of FIG. 8B. In a similar manner, FIG. 8E schematically shows a side view of the cell 128 of FIG. 8C. As can be seen from FIGS. 8B-8E, the cell 128 expands in the Z-direction, the X-direction, and the Y-direction. Accordingly, multiple cells 128 coupled together may form the triaxially expandable fabric portion 118. FIGS. 8B and 8D schematically show the cell 128 in the resting configuration 118R. FIGS. 8C and 8E schematically show the cell 128 in the stretched configuration 118S. For simplicity, the cells 128 of FIGS. 6B-6E are shown to describe the behavior of the triaxially expandable fabric portion 118. However, it should be understood that various embodiments are not limited to the shape of cells 128 shown here. Indeed, many different cell 128 shapes may be used. For example, each of the cells 128 may have an inward concave honeycomb shape. Furthermore, some embodiments may use structures 134 having a negative Poisson's ratio, such as auxetic yarn. It should also be understood that a variety of different materials and methods may be used to form the cells 128.

As described previously, the sub-components of the cell 128 (e.g., the joints 132 and/or the structures 134) may be formed from positive Poisson's ratio materials. However, the overall cell 128 and/or a fabric portion 118 may exhibit a negative Poisson's ratio. For example, the length and width of the cell 128 on FIG. 8B is increased in the stretched configuration shown in FIGS. 8D and 8E. However, the structures 134 themselves may have a decreased width relative to their increased length (e.g., as a normally positive Poisson's ratio stretched material behaves).

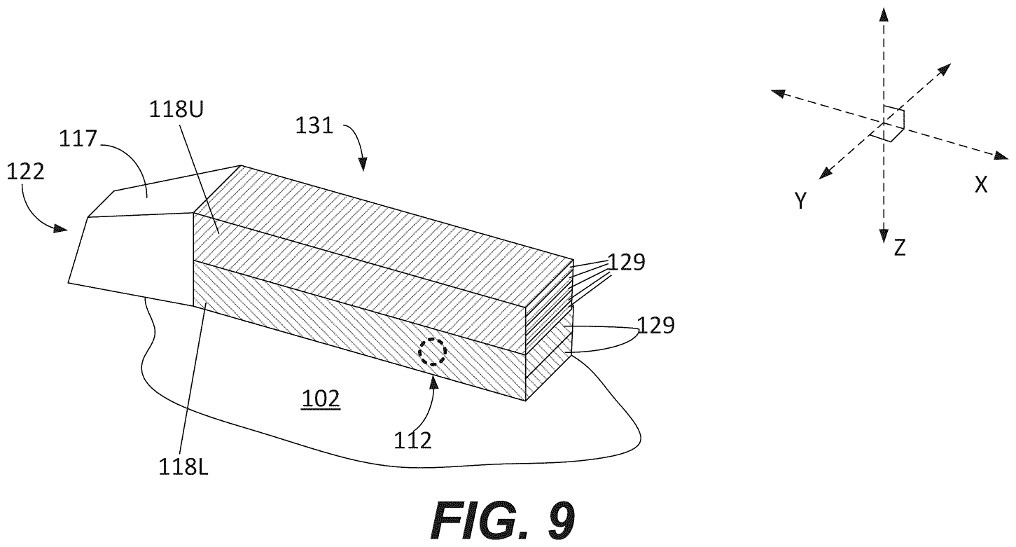

FIG. 9 schematically shows a multi-layer expandable fabric portion 131 in accordance with illustrative embodiments in this disclosure. The multi-layer expandable fabric portion 131 includes two or more multiaxially expandable fabric portions 118 coupled together. As described previously, each of the fabric portions 118 may have one or more cellular layers 129 configured to expand and/or contract the fabric portion along at least two perpendicular axes. In the example shown, the top layer expandable fabric portion 118 has five cellular layers 129 (shown only on a single face of the fabric portion 118U for discussion purposes) and the bottom layer expandable fabric layer 118 has two cellular layers 129 (again shown only on a single face of the fabric portion 118L for discussion purposes). Various embodiments may vary in cellular layer. For example, some fabric portions may have between 1 and 100 cellular layers 129.

The multi-layer expandable fabric portion 131 includes two fabric portions 118, an upper fabric portion 118U and a lower fabric portion 118L that at least partially overlap. The two fabric portions 118U and 118L are both multiaxially expandable, as discussed herein (e.g., biaxially expandable, triaxially expandable, etc.). However, the two fabric portions 118U and 118L may be configured to expand along different axes. For example, the lower fabric portion 118L may be configured to expand along the Y-axis (e.g., up and down on the skin of the patient 102) when stretched along the X-axis (e.g., circumferentially about the torso of the patient in an anatomical axial plane). The upper fabric portion 118U may be configured to expand along the Z-axis (e.g., into the patient 102) when stretched along the X-axis. Of course, one skilled in the art can envision a number of other multiaxially expandable arrangements other than those discussed in the above example.

In some embodiments, the multi-layer expandable fabric portion 131 may include an opening for the sensing electrode 112 and/or the therapy electrodes 114. Continuing with the example from the previous paragraph, a garment 110 including the multi-layer expandable fabric portion 131 may advantageously provide more natural and/or comfortable movement to the patient 102, with a reduced likelihood of the sensing electrodes 112 or therapy electrodes 114 mispositioning and/or flipping.

For example, as the patient 102 moves, the multi-layer expandable fabric portion 131 expands and/or contracts to maintain, or minimize deviation from, a desirable position of the electrode 112 relative to the patient 102. This may be accomplished by the expansion and/o contraction of the lower fabric portion 118L along multiple axes. Additionally, as the patient 102 moves, the multi-layer expandable fabric portion 131 expands and/or contracts to maintain, or minimize separation from, a desirable contact of the electrode 112 with the patient 102. This may be accomplished by the expansion and/or contraction of the upper fabric portion 118U along multiple axes.

Furthermore, illustrate embodiments may advantageously localize the stretch of the garment 110 to multiaxially expandable fabric portions 118U and/or 118L. For example, when the multi-layer expandable fabric portion 131 is formed as part of the belt 122, the expandable fabric portions 118 may be configured to resist stress less than the first fabric portion 117. Accordingly, in some embodiments, the smart garment 110 may be configured to reliably expand primarily along the multiaxially expandable fabric portions 131, before substantial compensatory expansion in the first fabric portion 117.

It should be further understood that although the fabric portions 118U and 118L are coupled together, the cells 128 of each portion 118U and 118L may move at least partially independently of one another. However, because the layers 118U and 118L are coupled, a stretch in one layer 118L (e.g., along the X-axis) may also cause a stretch in the other layer 118U (e.g., along the X-axis). The layers 118U and 118L may thus be expandable coupled, even if not expandable along identical axes (e.g., a stretch in the X-direction may cause the portion 118L to expand in the Y-direction, which stretches the portion 118U to expand in the Z-direction).

Although FIG. 9 refers to electrodes 112 and the belt 122, it should be understood that various embodiments may include the multi-layer expandable fabric portion 131 in other parts of the smart garment 110, including but not limited to portions adjacent to the therapy electrodes 114 (e.g., the pockets 56 and/or mesh 70) and other devices 110.

Additionally, although a two layer expandable fabric portion 131 is shown in FIG. 9, it should be understood that the multi-layer expandable fabric portion 131 has at least two layers of fabric portions 118. In some embodiments, the multi-layer expandable fabric portion 131 may have three layers, four layers, five layers, six layers, or more layers of fabric portions 131. Additionally, the fabric layers 118 are not limited to any particular shapes or axes shown in the above examples. For example, the fabric portion layers 118 may be circular in shape (e.g., see FIG. 4B).

In various embodiments, the smart garment 110 made include the multiaxially expandable fabric portion 118, for example, as part of the garment 110 and belt 122 shown in FIGS. 4A-4D. In particular, the smart garment 110 may be configured so that a stretch of the fabric portion 118 in the X-direction 109A is substantially a circumferential stretch (e.g., the patient's 102 torso expands against the belt 122, causing a circumferential stretch that stretches part of the belt 122 in the X-direction along the torso of the patient in an anatomical axial plane). Advantageously, the multiaxially expandable fabric portion 118 may be configured to expand in the Z-direction 109C (e.g., inwardly towards the patient 102) so as to keep the ECG electrodes 112 tighter to the skin. In a similar manner, portions of the garment 110 adjacent to the therapy electrodes 114 may be formed from the multiaxially expandable material 118 so as to keep the therapy electrodes 114 tight against the skin during stretch. For example, as shown in FIGS. 4D, the back of the garment 110 behind the pockets 56 may be formed as multiaxially expandable material portion 118. Additionally, or alternatively, the pockets 56 may be formed from the multiaxially expandable material portion 118. When stretched, the individual cells 128 of the pocket 56 may expand to provide a larger opening that facilitates passage of a gel expelled by the therapy electrode 114 to the patient 102.

As another example, the multiaxially expandable fabric portion 118 may be configured to stretch in the X-direction 109A and the Y-direction 109B so as to keep the electrodes 112 and/or 114 from shifting laterally. Some embodiments may additionally have two layers of expandable fabric portion 118, so that a stretch in one layer keeps the electrode 112 and/or 114 in a desired lateral position, while a stretch in the other layer pushes the electrode 112 and/or 114 towards the skin.

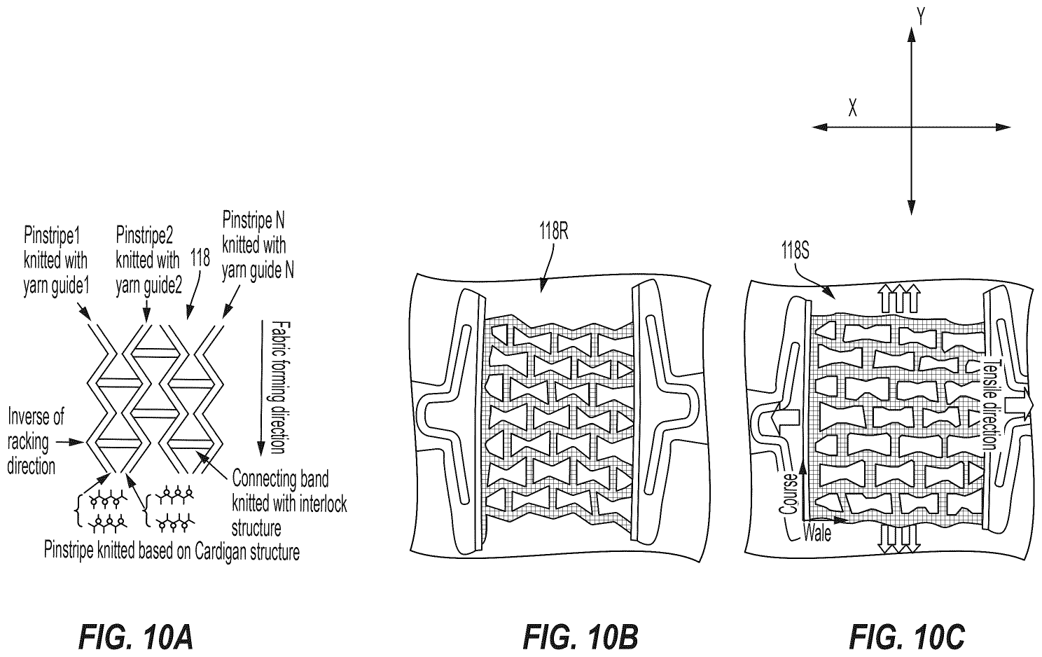

FIG. 10A schematically shows a knitting pattern for a multiaxially expandable fabric portion 118 in accordance with illustrative embodiments. FIG. 10B shows a picture of the knitted fabric portion 118 in the resting configuration 118R. FIG. 10C shows a picture of the knitted fabric portion 118 in the stretched configuration 118S. The multiaxially expandable fabric portion 118 includes reentrant hexagonal cells 128. As shown, the fabric portion 118 may be knitted using a plurality of yarn guides (e.g., yarn guide 1 to yarn guide N) in a fabric forming direction. 24/2 Nm 100% acrylic yarn was used to knit the whole fabric portion 118 shown in FIGS. 10B and 10C. The zigzag shape for each pinstripe may be formed using the racking process based on the Cardigan structure. The intarsia technique may be used to knit each separated pinstripe with an individual yarn carrier. The interlock structure may be used to connect the two neighboring pinstripes. The binding-off technique may be used to close the last knitting course of each connecting band. The knitted fabric exhibits the multiaxially expandable effect when extended in the fabric forming direction, as shown in FIGS. 10B and 10C.

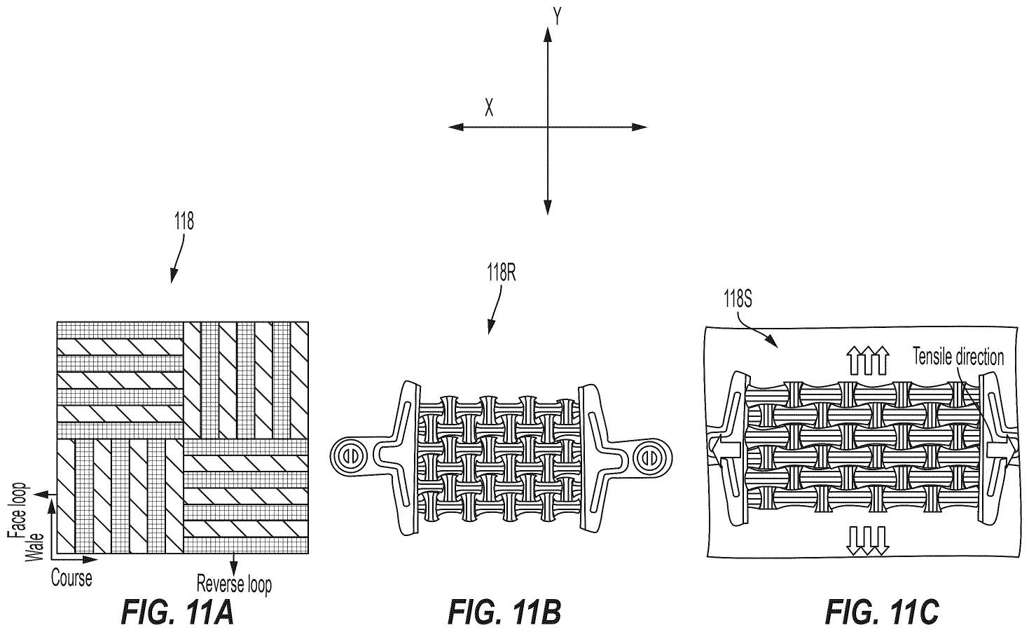

FIG. 11A schematically shows a knitting pattern for another multiaxially expandable fabric portion 118 formed with an arrangement of face and reverse loops in horizontal and vertical stripes. FIGS. 11B and 11C show the fabric portion 118 in the resting configuration 118R and the stretched configuration 118S, respectively. 30/2 Nm 100% mercerized wool yarn was used to knit the fabrics shown in FIGS. 11B and 11C. The multiaxially expandable effect can be obtained in two principal directions (e.g., the X-direction and the Y-direction). Indeed, in some embodiments, the multiaxially expandable fabric portion 118 may contract in one direction while expanding in two or more other directions. The multiaxially expandable effect of this fabric was achieved in a smaller range of the axial strain (45% in the course direction and 62% in the wale direction) because of less folded effect produced in this fabric. It can be found that the multiaxially expandable effect when extended in the course direction is higher than that when extended in the wale direction. The reason is that the stripes along the wale direction are closer than in the course direction, which increases more transverse expansion effect when extended along the course direction. It can also be found that the multiaxially expandable effects decrease with an increase of the strain for both directions. This is because with an increase of loading, the axial strain increase is faster than in the transverse strain due to the yarn transfer from the transverse direction to the axial direction.

As described above, the teachings of the present disclosure can be generally applied to externally worn, ambulatory medical monitoring and/or treatment smart garments 110. Such garments 110 may include devices 100 that are not completely implanted within the patient's 102 body. The smart garment 110 can include, for example, ambulatory devices 100 that are capable of and designed for moving with the patient 102 as the patient goes about his or her daily routine. An example ambulatory smart garment 110 can be a wearable, such as a wearable cardioverter defibrillator (WCD), a wearable cardiac monitoring device, an in-hospital device such as an in-hospital wearable defibrillator, a short-term wearable cardiac monitoring and/or therapeutic device, mobile telemetry devices, and other similar wearable garments.

The wearable can be capable of continuous use by the patient. In some implementations, the continuous use can be substantially or nearly continuous in nature. That is, the wearable may be continuously used, except for sporadic periods during which the use temporarily ceases (e.g., while the patient bathes, while the patient is refit with a new and/or a different garment, while the battery is charged/changed, while the garment is laundered, etc.). Such substantially or nearly continuous use as described herein may nonetheless qualify as continuous use. For example, the wearable can be configured to be worn by a patient for as many as 24 hours a day without substantial interruption. In some implementations, the patient may remove the wearable for a short portion of the day (e.g., for half an hour to bathe).

Further, the wearable smart garment 110 can be configured as a long term or extended use. Such garments 110 can be configured to be used by the patient, continuously, on a daily basis, for an extended period of several days, weeks, months, or even years. In some examples, the wearable can be used by a patient, continuously, on a daily basis, for an extended period of at least one week. In some examples, the wearable can be used by a patient, continuously, on a daily basis, for an extended period of at least 30 days. In some examples, the wearable garment 110 can be used by a patient, continuously, on a daily basis, for an extended period of at least one month. In some examples, the wearable garment 110 can be used by a patient, continuously, on a daily basis, for an extended period of at least two months. In some examples, the wearable garment 110 can be used by a patient, continuously, on a daily basis, for an extended period of at least three months. In some examples, the wearable garment 110 can be used by the patient, continuously, on a daily basis, for an extended period of at least six months. In some examples, the wearable garment 110 can be used by a patient, continuously, on a daily basis, for an extended period of at least one year. In some implementations, the extended use can be uninterrupted until a physician or other caregiver provides specific instruction to the patient to stop use of the wearable garment 110.

Regardless of the extended period of wear, the use of the wearable garment 110 can include continuous or nearly continuous wear by the patient as described above. For example, the continuous use can include continuous wear or attachment of the wearable garment 110 to the patient, e.g., through one or more of the electrodes as described herein, during both periods of monitoring and periods when the device 100 may not be monitoring the patient but is otherwise still worn by or otherwise attached to the patient. The wearable garment 110 can be configured to continuously monitor the patient for cardiac-related information (e.g., electrocardiogram (ECG) information, including arrhythmia information, heart vibrations, etc.) and/or non-cardiac information (e.g., blood oxygen, the patient's temperature, glucose levels, tissue fluid levels, and/or lung vibrations). The wearable garment 110 can carry out its monitoring in periodic or aperiodic time intervals or times. For example, the monitoring during intervals or times can be triggered by a user action or another event.

As noted above, the wearable garment 110 can be configured to monitor other physiologic parameters of the patient in addition to cardiac related parameters. For example, the wearable can be configured to monitor, for example, lung vibrations (e.g., using microphones and/or accelerometers), breath vibrations, sleep related parameters (e.g., snoring, sleep apnea), tissue fluids (e.g., using radio-frequency transmitters and sensors), among others.

Other example wearable garments 110 include automated cardiac monitors and/or defibrillators for use in certain specialized conditions and/or environments such as in combat zones or within emergency vehicles. Such devices can be configured so that they can be used immediately (or substantially immediately) in a life-saving emergency. In some examples, the wearable garments 110 described herein can be pacing-enabled, e.g., capable of providing therapeutic pacing pulses to the patient.

In some implementations, the wearable ambulatory device may be operated in patient monitoring mode device where the treatment and/or therapy functions are removed/deactivated. For example, such a wearable ambulatory garment 110 can be configured to monitor one or more cardiac physiological parameters of a patient, e.g., for remotely monitoring and/or diagnosing a condition of the patient. For example, such cardiac physiological parameters may include a patient's ECG information, heart vibrations (e.g., using accelerometers or microphones), and other related cardiac information. In this regard, the wearable ambulatory garment 110 is configured to detect the patient's ECG through a plurality of cardiac sensing electrodes 112. Example cardiac conditions can include atrial fibrillation, bradycardia, tachycardia, atrio-ventricular block, Lown-Ganong-Levine syndrome, atrial flutter, sino-atrial node dysfunction, cerebral ischemia, syncope, atrial pause, and/or heart palpitations. When such an anomaly is detected, the monitor may automatically send data relating to the anomaly to a remote server. The remote server may be located within a 24-hour manned monitoring center, where the data is interpreted by qualified, cardiac-trained reviewers and/or caregivers, and feedback provided to the patient and/or a designated caregiver via detailed periodic or event-triggered reports. In certain cardiac event monitoring applications, the cardiac monitor is configured to allow the patient to manually press a button on the cardiac monitor (e.g., on the patient interface pod 140) to report a symptom. For example, a patient may report symptoms such as a skipped beat, shortness of breath, light headedness, racing heart rate, fatigue, fainting, chest discomfort, weakness, dizziness, and/or giddiness. The wearable ambulatory device can record predetermined physiologic parameters of the patient (e.g., ECG information) for a predetermined amount of time (e.g., 1-30 minutes before and 1-30 minutes after a reported symptom). The wearable ambulatory device can be configured to monitor physiologic parameters of the patient other than cardiac related parameters. For example, the wearable ambulatory device can be configured to monitor, for example, heart vibrations (e.g., using accelerometers or microphones), lung vibrations, breath vibrations, sleep related parameters (e.g., snoring, sleep apnea), tissue fluids, among others.

FIG. 12 schematically illustrates a sample component-level view of the controller 120. As shown in FIG. 12, the smart garment controller 120 can include a therapy delivery circuit 202, a data storage 204, a network interface 206, a user interface 208, at least one battery 210, a sensor interface 212, an alarm manager 214, and at least one processor 218. A patient monitoring smart garment 110 can include a controller 120 that includes like components as those described above, but does not include the therapy delivery circuit 202 (shown in dotted lines).

The therapy delivery circuit 202 can be coupled to one or more electrodes 220 configured to provide therapy to the patient (e.g., therapy electrodes 114 as described above). For example, the therapy delivery circuit 202 can include, or be operably connected to, circuitry components that are configured to generate and provide the therapeutic shock. The circuitry components can include, for example, resistors, capacitors, relays and/or switches, electrical bridges such as an h-bridge (e.g., including a plurality of insulated gate bipolar transistors or IGBTs), voltage and/or current measuring components, and other similar circuitry components arranged and connected such that the circuitry components work in concert with the therapy delivery circuit and under control of one or more processors (e.g., processor 218) to provide, for example, one or more pacing or defibrillation therapeutic pulses.

Pacing pulses can be used to treat cardiac arrhythmias such as bradycardia (e.g., less than 30 beats per minute) and tachycardia (e.g., more than 100 beats per minute) using, for example, fixed rate pacing, demand pacing, anti-tachycardia pacing, and the like. Defibrillation pulses can be used to treat ventricular tachycardia and/or ventricular fibrillation.

The capacitors can include a parallel-connected capacitor bank consisting of a plurality of capacitors (e.g., two, three, four or more capacitors). These capacitors can be switched into a series connection during discharge for a defibrillation pulse. For example, four capacitors of approximately 650 uF can be used. The capacitors can have between 350 to 500 volt surge rating and can be charged in approximately 15 to 30 seconds from a battery pack.

For example, each defibrillation pulse can deliver between 25 and 400 joules of energy (e.g., between 60 to 180 joules) of energy. In some implementations, the defibrillating pulse can be a biphasic truncated exponential waveform, whereby the signal can switch between a positive and a negative portion (e.g., charge directions). This type of waveform can be effective at defibrillating patients at lower energy levels when compared to other types of defibrillation pulses (e.g., such as monophasic pulses). For example, an amplitude and a width of the two phases of the energy waveform can be automatically adjusted to deliver a precise energy amount (e.g., 150 joules) regardless of the patient's body impedance. The therapy delivery circuit 202 can be configured to perform the switching and pulse delivery operations, e.g., under control of the processor 218. As the energy is delivered to the patient, the amount of energy being delivered can be tracked. For example, the amount of energy can be kept to a predetermined constant value even as the pulse waveform is dynamically controlled based on factors such as the patient's body impedance which the pulse is being delivered.

The data storage 204 can include one or more of non-transitory computer readable media, such as flash memory, solid state memory, magnetic memory, optical memory, cache memory, combinations thereof, and others. The data storage 204 can be configured to store executable instructions and data used for operation of the controller 120. In certain implementations, the data storage can include executable instructions that, when executed, are configured to cause the processor 218 to perform one or more functions.

In some examples, the network interface 206 can facilitate the communication of information between the controller 120 and one or more other devices or entities over a communications network. For example, where the controller 120 is included in an ambulatory (such as smart garment 110), the network interface 206 can be configured to communicate with a remote computing device such as a remote server or other similar computing device. The network interface 206 can include communications circuitry for transmitting data in accordance with a Bluetooth® wireless standard for exchanging such data over short distances to an intermediary device(s) (e.g., a base station, a “hotspot” device, a smartphone, a tablet, a portable computing device, and/or other devices in proximity of the wearable smart garment 110). The intermediary device(s) may in turn communicate the data to a remote server over a broadband cellular network communications link. The communications link may implement broadband cellular technology (e.g., 2.5G, 2.75G, 3G, 4G, 5G cellular standards) and/or Long-Term Evolution (LTE) technology or GSM/EDGE and UMTS/HSPA technologies for high-speed wireless communication. In some implementations, the intermediary device(s) may communicate with a remote server over a Wi-Fi™ communications link based on the IEEE 802.11 standard.

In certain implementations, the user interface 208 can include one or more physical interface devices such as input devices, output devices, and combination input/output devices and a software stack configured to drive operation of the devices. These user interface elements may render visual, audio, and/or tactile content. Thus the user interface 208 may receive input or provide output, thereby enabling a user to interact with the controller 120.

The controller 120 can also include at least one battery 210 configured to provide power to one or more components integrated in the controller 120. The battery 210 can include a rechargeable multi-cell battery pack. In one example implementation, the battery 210 can include three or more 2200 mAh lithium ion cells that provide electrical power to the other device components within the controller 120. For example, the battery 210 can provide its power output in a range of between 20 mA to 1000 mA (e.g., 40 mA) output and can support 24 hours, 48 hours, 72 hours, or more, of runtime between charges. In certain implementations, the battery capacity, runtime, and type (e.g., lithium ion, nickel-cadmium, or nickel-metal hydride) can be changed to best fit the specific application of the controller 120.

The sensor interface 212 can be coupled to one or more sensors configured to monitor one or more physiological parameters of the patient. As shown, the sensors may be coupled to the controller 120 via a wired or wireless connection. The sensors can include one or more electrocardiogram (ECG) electrodes 112 (e.g., similar to sensing electrodes 112 as described above), heart vibrations sensors 224, and tissue fluid monitors 226 (e.g., based on ultra-wide band radiofrequency devices).

The ECG electrodes 112 can monitor a patient's ECG information. For example, the ECG electrodes 112 can be galvanic (e.g., conductive) and/or capacitive electrodes configured to measure changes in a patient's electrophysiology to measure the patient's ECG information. The ECG electrodes 112 can transmit information descriptive of the ECG signals to the sensor interface 212 for subsequent analysis.

The heart vibrations sensors 224 can detect a patient's heart vibration information. For example, the heart vibrations sensors 224 can be configured to detect heart vibration values including any one or all of S1, S2, S3, and S4. From these heart vibration values, certain heart vibration metrics may be calculated, including any one or more of electromechanical activation time (EMAT), percentage of EMAT (% EMAT), systolic dysfunction index (SDI), and left ventricular systolic time (LVST). The heart vibrations sensors 224 can include an acoustic sensor configured to detect vibrations from a subject's cardiac system and provide an output signal responsive to the detected heart vibrations. The heart vibrations sensors 224 can also include a multi-channel accelerometer, for example, a three channel accelerometer configured to sense movement in each of three orthogonal axes such that patient movement/body position can be detected and correlated to detected heart vibrations information. The heart vibrations sensors 224 can transmit information descriptive of the heart vibrations information to the sensor interface 212 for subsequent analysis.

The tissue fluid monitors 226 can use radio frequency (RF) based techniques to assess fluid levels and accumulation in a patient's body tissue. For example, the tissue fluid monitors 226 can be configured to measure fluid content in the lungs, typically for diagnosis and follow-up of pulmonary edema or lung congestion in heart failure patients. The tissue fluid monitors 226 can include one or more antennas configured to direct RF waves through a patient's tissue and measure output RF signals in response to the waves that have passed through the tissue. In certain implementations, the output RF signals include parameters indicative of a fluid level in the patient's tissue. The tissue fluid monitors 226 can transmit information descriptive of the tissue fluid levels to the sensor interface 212 for subsequent analysis.

The sensor interface 212 can be coupled to any one or combination of sensing electrodes/other sensors to receive other patient data indicative of patient 102 parameters. Once data from the sensors has been received by the sensor interface 212, the data can be directed by the processor 218 to an appropriate component within the controller 120. For example, if heart data is collected by heart vibrations sensor 224 and transmitted to the sensor interface 212, the sensor interface 212 can transmit the data to the processor 218 which, in turn, relays the data to a cardiac event detector. The cardiac event data can also be stored on the data storage 204.

In certain implementations, the alarm manager 214 can be configured to manage alarm profiles and notify one or more intended recipients of events specified within the alarm profiles as being of interest to the intended recipients. These intended recipients can include external entities such as users (patients, physicians, and monitoring personnel) as well as computer systems (monitoring systems or emergency response systems). The alarm manager 214 can be implemented using hardware or a combination of hardware and software. For instance, in some examples, the alarm manager 214 can be implemented as a software component that is stored within the data storage 204 and executed by the processor 218. In this example, the instructions included in the alarm manager 214 can cause the processor 218 to configure alarm profiles and notify intended recipients using the alarm profiles. In other examples, alarm manager 214 can be an application-specific integrated circuit (ASIC) that is coupled to the processor 218 and configured to manage alarm profiles and notify intended recipients using alarms specified within the alarm profiles. Thus, examples of alarm manager 214 are not limited to a particular hardware or software implementation.

In some implementations, the processor 218 includes one or more processors (or one or more processor cores) that each are configured to perform a series of instructions that result in manipulated data and/or control the operation of the other components of the controller 120. In some implementations, when executing a specific process (e.g., cardiac monitoring), the processor 218 can be configured to make specific logic-based determinations based on input data received, and be further configured to provide one or more outputs that can be used to control or otherwise inform subsequent processing to be carried out by the processor 218 and/or other processors or circuitry with which processor 218 is communicatively coupled. Thus, the processor 218 reacts to specific input stimulus in a specific way and generates a corresponding output based on that input stimulus. In some example cases, the processor 218 can proceed through a sequence of logical transitions in which various internal register states and/or other bit cell states internal or external to the processor 218 may be set to logic high or logic low. As referred to herein, the processor 218 can be configured to execute a function where software is stored in a data store coupled to the processor 218, the software being configured to cause the processor 218 to proceed through a sequence of various logic decisions that result in the function being executed. The various components that are described herein as being executable by the processor 218 can be implemented in various forms of specialized hardware, software, or a combination thereof. For example, the processor can be a digital signal processor (DSP) such as a 24-bit DSP processor. The processor can be a multi-core processor, e.g., having two or more processing cores. The processor can be an Advanced RISC Machine (ARM) processor such as a 32-bit ARM processor. The processor can execute an embedded operating system, and include services provided by the operating system that can be used for file system manipulation, display & audio generation, basic networking, firewalling, data encryption and communications.

Although the subject matter contained herein has been described in detail for the purpose of illustration, it is to be understood that such detail is solely for that purpose and that the present disclosure is not limited to the disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

Other examples are within the scope and spirit of the description and claims. Additionally, certain functions described above can be implemented using software, hardware, firmware, hardwiring, or combinations of any of these. Features implementing functions can also be physically located at various positions, including being distributed such that portions of functions are implemented at different physical locations.

1. A non-invasive, wearable, ambulatory device capable of cardiac defibrillation comprising:
a plurality of ECG electrodes and associated ECG circuitry configured to sense ECG signals from a patient;
a plurality of therapy electrodes configured to deliver one or more defibrillation pulses to the patient; and
a smart garment configured to be worn around a torso of the patient, the smart garment comprising:
a first fabric portion; and
a multiaxially expandable fabric portion having a first axis and a second axis, the multiaxially expandable fabric portion configured to expand along the first axis as the multiaxially expandable fabric portion expands along the second axis, the multiaxially expandable fabric portion being coupled with the first fabric portion, the first axis and the second axis being substantially normal to one another;
wherein the plurality of ECG electrodes are coupled with the multiaxially expandable portion, the first fabric portion, or both the multiaxially expandable fabric portion and the first fabric portion,
wherein the smart garment is configured to maintain continuous electrical contact between the plurality of ECG electrodes and skin of the patient over a duration of time when the smart garment is worn about the torso of the patient.

2. The device of claim 1, wherein the smart garment is configured to maintain continuous electrical contact between the plurality of therapy electrodes and skin of the patient over the duration of time when the smart garment is worn about the torso of the patient.

3. The device of claim 1, wherein the plurality of ECG comprises one or more non-adhesive ECG electrodes, dry ECG electrodes. and/or polarizable ECG electrodes.

4-5. (canceled)

6. The device of claim 1, further comprising a controller configured to detect:
ventricular fibrillation (“VF”), and/or
ventricular tachycardia (“VT”); and
further configured to deliver one or more defibrillation pulses to the patient via the plurality of therapy electrodes as a function of detecting.

7-8. (canceled)

9. The device of claim 6, wherein energy of one or more of the defibrillation pulses is between 25 and 400 joules.

10. The device of claim 6, wherein the pulses are cardioversion pulses, and wherein the energy of one or more of the cardioversion pulses is between 25 and 400 joules.

11. The device of claim 1, further comprising a controller configured to detect one or more of bradycardia, tachycardia, or asystole, and to deliver one or more pacing pulses to the patient via the plurality of therapy electrodes.

12. (canceled)

13. The device of claim 11, wherein current of one or more of the pacing pulses is between 0.1 mA and 300 mA.

14. (canceled)

15. The device of claim 1, wherein the smart garment is configured to maintain the electrical contact between one or more of the plurality of ECG electrodes and skin of the patient at least by pressing the one or more of the plurality of ECG electrodes against the skin of the patient of the smart garment at a predetermined range of between about 0.1 psi and about 3 psi.

16. (canceled)

17. The device of claim 1, further comprising a controller configured to trigger an audible alarm when an arrhythmia is detected.

18-22. (canceled)

23. The device of claim 1, wherein the plurality of ECG electrodes are removably coupled with the smart garment.

24. (canceled)

25. The device of claim 1, wherein the multiaxially expandable fabric portion comprises a matrix formed of a plurality of single cells, each of the single cells having a predetermined shape.

26-40. (canceled)

41. The device of claim 1, wherein the multiaxially expandable fabric portion comprises a fiber-based non-conductive yarn having a multiaxially expandable fabric structural unit.

42-50. (canceled)

51. A smart garment for use in monitoring, the smart garment comprising:
a first fabric portion; and
a multiaxially expandable fabric portion, the multiaxially expandable portion having a first axis and a second axis normal to one another, the multiaxially expandable fabric portion configured to expand along the first axis as the multiaxially expandable fabric portion expands along the second axis,
the first fabric portion being coupled with the multiaxially expandable fabric portion,
one or more electrodes coupled with the multiaxially expandable fabric portion, the first fabric portion, or both the multiaxially expandable fabric portion and the first fabric portion.

52. The smart garment of claim 51, wherein the first fabric portion is configured to cause the smart garment to maintain electrical contact between the one or more ECG electrodes and skin of a patient when the smart garment is subject to forces that cause the smart garment to either one of (a) stretch along, or (b) twist about, a circumference of the patient's body.

53. The smart garment of claim 51, wherein the multiaxially expandable fabric portion is an auxetic material.

54. The smart garment of claim 51, wherein the multiaxially expandable fabric portion comprises a multiaxially expandable fiber, multiaxially expandable fabric yarn, or both a multiaxially expandable fiber and a multiaxially expandable fabric yarn.

55. The smart garment of claim 54, wherein the multiaxially expandable fabric yarn comprises a double helix yarn.

56. The smart garment of claim 55, wherein a wrap material of the double helix yarn comprise an ultra-high molecular weight polyethylene fiber and a core material is polyurethane.

57. The smart garment of claim 51, wherein the multiaxially expandable fabric portion comprises a fiber-based dielectric yarn having a multiaxially expandable fabric structural unit.

58. The smart garment of claim 57, wherein the fiber-based yarn has a reentrant hexagonal structural unit in an unstretched configuration, and a honeycomb structural unit in a stretched configuration, wherein the fiber-based yarn is lengthened between 5% and 25% in the stretched position relative to the unstretched position.

59. The smart garment of claim 51, wherein the multiaxially expandable fabric portion is formed from a single layer multiaxially expandable fabric material.

60. The smart garment of claim 51, wherein the multiaxially expandable fabric portion is formed from a double layer multiaxially expandable fabric material.

61-80. (canceled)
